(19) United States
(12) Patent Application Publication
(10) Pub. No.: US 2013/0244965 A1
(43) Pub. Date: Sep. 19, 2013

(54) ASSAYS AND METHODS FOR DETERMINING RISK OF A MACROPHAGE-MEDIATED DISEASE DEVELOPMENT IN A SUBJECT INFECTED WITH HIV

(71) Applicants: Kenneth C. Williams, Newburyport, MA (US); Tricia Burdo, Shrewsbury, MA (US); Steven K. Grinspoon, Weston, MA (US); Eric Rosenberg, Milton, MA (US)

(72) Inventors: Kenneth C. Williams, Newburyport, MA (US); Tricia Burdo, Shrewsbury, MA (US); Steven K. Grinspoon, Weston, MA (US); Eric Rosenberg, Milton, MA (US)

(73) Assignees: THE GENERAL HOSPITAL CORPORATION, Boston, MA (US); TRUSTEES OF BOSTON COLLEGE, Chestnut Hill, MA (US)

(21) Appl. No.: 13/876,050

(22) PCT Filed: Sep. 27, 2011
(86) PCT No.: PCT/US2011/053427

§ 371 Date: May 30, 2013

Related U.S. Application Data
(60) Provisional application No. 61/387,226, filed on Sep. 28, 2010.

(51) Int. Cl. G01N 33/68

(52) U.S. Cl. CPC G01N 33/6893
USPC 514/45; 435/7.92; 435/287.2

(57) ABSTRACT

The invention is generally related to assays and methods for determining the risk of an HIV+ individual for developing a macrophage-mediated disease using measurement of soluble CD163 levels in a biological sample. The invention also provides assays and methods for monitoring efficacy of a treatment or a drug for a macrophage-mediated disease, and assays and methods for screening for agents to treat a macrophage-mediated disease in an HIV+ individual by monitoring soluble CD163 levels.

### CROSS-REFERENCE TO RELATED APPLICATIONS

This application claims benefit under 35 U.S.C. §119(e) of a provisional application No. 61/387,226, filed on Sep. 28, 2010, the content of which is herein incorporated by reference in its entirety.

### GOVERNMENT SUPPORT

This invention was made with Government support under Grant Nos. RO1NS40237 and RO1NS37654 awarded by the National Institutes of Health. The Government has certain rights in the invention.

### FIELD OF THE INVENTION

The field of the invention is related to the early determination of risk of HIV related disorders in subjects infected with HIV. The invention further provides for monitoring treatment efficacy and methods for screening agents to treat a macrophage-mediated disease, for example, in subjects infected with HIV.

### BACKGROUND OF THE INVENTION

Acquired immune deficiency syndrome (“AIDS”) is a disease caused by the human immunodeficiency virus (“HIV”). The main targets of HIV include the CD4+ T cell and the macrophage. Once infected the T4 lymphocyte population generally begins to decline; however, unlike the T4 lymphocytes, the macrophage is not killed by HIV infection but may actually serve as a reservoir for the virus. Gallo & Montagneir “AIDS in 1988”, Scientific American, p. 25 (October 1988).

The early diagnosis of persons suffering from HIV infection generally involves using diagnostic tests to determine whether or not a person has antibodies to HIV. Early diagnosis is especially important in HIV infection as it enables the patient to receive optimal medical care from the earliest moments of the disease and to check further spread of the contagion. Redfield & Burke “HIV Infection: The Clinical Picture”, Scientific American, p. 70.

HIV is known to target specific subpopulations of T-cells in the human body, which results in a severe immune deficiency of these patients due to an unusually low proportion of T-cells (T4) in their lymphocyte population. As a result the availability of many T4 helper functions is reduced, including e.g., the production of antibodies by the B-cells. Depressed cellular immunity in HIV+ individuals is also associated with serious opportunistic infections, cancers and other disorders, such as, AIDS-related dementia, peripheral neuropathy, and HIV-associated heart disease.

Given the repressed immune systems in HIV+ individuals, early diagnosis of HIV- or AIDS-related disorders is important so that appropriate treatment can be initiated at an early stage of disease, for example, even before the overt expression of clinical symptoms. It is also important to assess and monitor the level of risk for development of an HIV- or AIDS-related diseases and disorders in an HIV positive (HIV+) individual.

### SUMMARY OF THE INVENTION

The invention provides assays, systems, and methods for monitoring disease activity in individuals infected with HIV. Specifically, assays, systems, and methods are provided for monitoring macrophage-mediated disease activity, such as, peripheral neuropathy, HIV-associated heart disease, atherosclerosis, and AIDS-related dementia, in individuals infected with HIV. Provided herein are methods and assays for determining the risk of an HIV+ individual to develop an HIV-related or AIDS-related disorder mediated by macrophages. Also provided herein are methods and assays for monitoring risk of an HIV+ individual, as well as monitoring the efficacy of treatment of a macrophage-mediated disorder in HIV+ individuals. In some aspects of all the embodiments of the invention, the individual is either an HIV+ male or an HIV+ female individual. In some aspects of all the embodiments of the invention, the individual is an HIV+ male individual.

The methods and assays provided herein are based, in part, on the discovery that levels of soluble CD163 (sCD163) are increased in HIV+ individuals compared to HIV-serotype negative individuals. Further, the inventors have discovered that treatment of HIV+ individuals with antiretroviral drugs (ART) reduces the level of sCD163 compared to HIV+ individuals who are not treated with ART. The inventors have discovered that levels of sCD163 correlate with HIV-disease activity, for example, the sCD163 levels correlate with level of HIV replication in plasma and disease progression. Individuals infected with HIV, particularly HIV not successfully controlled using ART, are at a higher risk of developing HIV-related disorders, such as macrophage-mediated diseases. Thus, provided herein are assays and methods for determining the risk of an HIV+ individual for developing a macrophage-mediated disease. Also provided are assays and methods for monitoring efficacy of a treatment for a macrophage-mediated disease, and assays and methods for screening for agents to treat a macrophage-mediated disease in an HIV+ individual.

CD163 has been indicated as a marker for inflammation and inflammatory conditions in individuals who are not infected with HIV (U.S. Pat. No. 7,144,710). Moreover, the immune system is known to be severely affected and dysfunctional in individuals infected with HIV. Therefore, an inflammatory marker seen in non-HIV infected individuals was not expected to have value as a marker in HIV-infected individuals. Specifically, one could not have expected a marker that is highly expressed in normal macrophages to work similarly in HIV infected and HIV non-infected individuals because macrophages play a crucial role in HIV-1 infection. Macrophages are among the first cells infected by HIV-1, and have been proposed to form a reservoir of HIV-1 in infected persons.

Moreover, the inventors here describe that sCD163 can serve as a marker for dementia in HIV infected individuals but the association is not seen in individuals with dementia but not infected with HIV. Thus sCD163 provides a particularly valuable marker in monitoring HIV-associated dementia and provides an early marker that can be used prior to onset of the symptoms for this condition. Similarly, HIV-associated cardiomyopathy is particularly associated with sCD163 levels and can be detected early, even prior to the onset of the symptoms.

Accordingly, in one embodiment, the invention provides an assay for determining risk for onset of a macrophage-mediated disease in an individual infected with HIV, the assay comprising the steps of: (a) contacting a first biological sample, wherein the first biological sample comprises plasma, blood, or cerebral spinal fluid obtained from the individual at a first time point with an antibody against soluble CD163; (b) measuring the amount of soluble CD163 in the first biological sample; (c) contacting at least a second biological sample, wherein the second biological sample comprises plasma, blood, or cerebral spinal fluid obtained from the individual at a subsequent time point with an antibody against the soluble CD163; (d) measuring the amount of the soluble CD163 in the at least second biological sample; and (e) comparing the difference in the amount of the soluble CD163 between the first and the at least second biological sample, wherein the individual is at risk for onset of a macrophage-mediated disease if the amount of soluble CD163 is increased by at least 10% in the at least second biological sample. In some aspects of this and all the other embodiments of the invention, the antibody against the soluble CD163 is an antibody specifically recognizing the extracellular domain of CD163. In some aspects of this and all the other embodiments of the invention, the antibody against the soluble CD163 is an antibody specifically recognizing the C-terminal end of the soluble CD163 protein.

In another embodiment, the invention provides an assay for determining or monitoring the effectiveness of a treatment of a macrophage-mediated disease in an individual infected with HIV, comprising the steps of: (a) contacting a first biological sample, wherein the first biological sample comprises plasma, blood or cerebral spinal fluid that is obtained from an individual infected with HIV and further infected with a macrophage-mediated disease prior to administering a treatment with an antibody against the soluble CD163; (b) measuring the amount of the soluble CD163 in the first biological sample; (c) administering the treatment to the patient; (d) contacting a second biological sample, wherein the second biological sample comprises plasma, blood, or cerebral spinal fluid from the individual obtained after administration of the treatment with an antibody against the soluble CD163; (e) measuring the amount of the soluble CD163 in the second biological sample; and (f) comparing the difference in the amount of the soluble CD163 between the first and the second biological sample, wherein the treatment is effective if the amount of soluble CD163 in the second biological sample is decreased by at least 10% compared to the first biological sample.

In some aspects of this embodiment and the other embodiments of the invention, the assay further comprises administering to the individual a different treatment or increased dosage of the same treatment if the amount of the soluble CD163 is not decreased by at least 10% in the second biological sample.

In another embodiment, the invention provides an assay for screening for an agent to treat a macrophage-mediated disease in a test model infected with HIV or SIV, comprising the steps of: (a) contacting a first biological sample, wherein the first biological sample comprises plasma, blood, or cerebral spinal fluid obtained from a test model infected with HIV or SIV and further affected with a macrophage-mediated disease prior to administering a treatment to the test model with an antibody against the soluble CD163; (b) measuring the amount of the soluble CD163 in the first biological sample; (c) administering the treatment to the test model; (d) contacting a second biological sample comprising plasma, blood, or cerebrospinal fluid from the test model obtained from the test model after administering the treatment with an antibody against the soluble of CD163; (e) measuring the amount of the soluble CD163 in the second biological sample; and (d) comparing the difference in the amount of the soluble CD163 between the first and second biological sample, wherein the agent is effective to treat a macrophage-mediated disease if the amount of the soluble CD163 in the second biological sample is decreased by at least 10% compared to the first biological sample.

In another embodiment, the invention provides an assay for determining risk of HIV infection in an individual who has not undergone seroconversion, comprising the steps of: (a) contacting a first biological sample comprising plasma, blood, or cerebral spinal fluid from the individual with an antibody against the soluble CD163 and measuring the amount of the soluble CD163 in the first biological sample; (b) contacting a second biological sample comprising plasma, blood, or cerebral spinal fluid with an antibody against the soluble CD163 and measuring the amount of the soluble CD163 in the second biological sample, and (c) determining the difference in the amount of the soluble CD163 between the first and second biological sample, wherein the individual is at risk for HIV infection if the amount of the soluble CD163 is increased by at least 10% in the second biological sample compared to the first biological sample.

The invention further provides a method for determining risk of HIV infection in an individual who has not undergone seroconversion, comprising the steps of: (a) transforming a first and second biological sample comprising blood from an individual infected with HIV to deplete the first and second biological sample of monocytes; (b) contacting a first biological sample comprising plasma, blood, or cerebral spinal fluid from the individual with an antibody against the soluble CD163 and measuring the amount of the soluble CD163 in the first biological sample; (c) contacting a second biological sample comprising plasma, blood, or cerebral spinal fluid with an antibody against the soluble CD163 and measuring the amount of the soluble CD163 in the second biological sample; and (d) determining the difference in the amount of the soluble CD163 between the first and second biological sample, wherein the individual is at risk for HIV infection if the amount of the soluble CD163 is increased by at least 10% in the second biological sample compared to the first biological sample.

In some aspects of the assays and methods of the invention, the macrophage-mediated disease is selected from the group consisting of: AIDS-related dementia, peripheral neuropathy, and HIV-associated heart disease. In some aspects the HIV-associated heart disease is HIV-associated cardiomyopathy.

In some aspects, the assay of any embodiment of the invention further comprises the step of depleting the first and/or the at least second biological sample from monocytes.

In some aspects of any embodiment of the invention, the individual has detectable HIV levels.

In some aspects of any embodiment of the invention, the second biological sample is obtained at least about 1, 2, 3, 4, 5, 6, 7, 8, 9, 10, or 11 weeks after the first biological sample.

In some aspects of any embodiment of the invention, the second biological sample is obtained at least 3 months after the first biological sample.

In some aspects of any embodiment of the invention, the second biological sample is obtained at least 6 months after the first biological sample.

In some aspects of any embodiment of the invention, the test model is a mammalian model. The mammalian model can be a primate model or a human. In some aspects, the mammalian model is a non-human mammal.

In some aspects of any of the embodiments, the measuring step is performed using an ELISA technique.

In some aspects of any of the embodiments, the antibody specifically differentiates between the soluble and macrophage-associated CD163, and does not bind to macrophage-associated CD163.

In some aspects of any of the embodiments, the second biological sample is obtained at least 9 months after the first biological sample.

In yet another embodiment, the invention provides a method for determining time of onset for administering treatment to an individual infected with HIV prior to appearance of clinical symptoms, the method comprising the steps of: (a) contacting a first biological sample, wherein the first biological sample comprises plasma, blood, or cerebral spinal fluid obtained from the individual with an antibody against the soluble CD163; (b) measuring the amount of the soluble CD163 in the first biological sample; (c) comparing the amount of the soluble CD163 in the first biological sample to a reference; (d) administering a treatment to the individual if the amount of the soluble CD163 is increased by at least 10% in the first biological sample compared to the reference.

In some aspects of the embodiments, the reference is a second biological sample, wherein the second biological sample comprises plasma, blood, or cerebral spinal fluid obtained from the individual at a time point that is subsequent to obtaining the first biological sample.

In some embodiments, the reference is a numerical value comprising the normal range of soluble CD163 in an individual. In some embodiments, the reference is a normal value or range of values in HIV infected individuals who do not have the condition that is to be monitored. In some embodiments, the normal value or range of values in individuals who are not infected with HIV and do not have the conditions to be determined, such as heart disease, e.g., cardiomyopathy, dementia, or atherosclerosis.

### DEFINITIONS

As used herein, the term “sCD163” refers to the CD163 that is shed from macrophages/monocytes. The sDC163 contains the extracellular domain of CD163, for example, in response to activation by e.g., LPS stimulation or retroviral infection. The CD163 does not contain the intracellular or the intact transmembrane domains of the CD163. The sCD163 is therefore different from the macrophage-associated CD163.

As used herein, the term “macrophage-mediated disease” refers to any disease that is caused by or associated with an increase in macrophage number or macrophage activation in HIV+ individuals or in SIV+ primates. Some non-limiting examples of macrophage-mediated diseases include AIDS-associated dementia, HIV-associated heart disease (e.g., coronary atherosclerosis), and peripheral neuropathy.

As used herein, the phrase “individual infected with HIV” refers to an individual that has been diagnosed as having HIV by e.g., detecting the presence of HIV antibodies in a sample obtained from the individual. An individual infected with HIV can also have symptoms of AIDS. Diagnosis of HIV can be performed by any method known in the clinic, in the art, or as described herein.

As used herein, the phrase “individual who has not undergone seroconversion” refers to an individual at risk of having HIV infection but has not yet developed HIV antibodies detectable using standard techniques (e.g., ELISA or Western blot). The individual may be a male or a female individual. In some embodiments, the individual is a male individual.

As used herein, the phrase “obtained from an individual” encompasses samples that need not be directly assayed and can be e.g., stored, transported, transformed, or treated as necessary to produce a workable sample, for example by depleting monocytes from the sample for detection of soluble CD163 (e.g., preparation of plasma or serum from whole blood) in the absence of cross-contamination with membrane-associated CD163.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies, camelid and heavy chain antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage using methods known to those of skill in the art. The term further encompasses an antibody which has been generated by the synthesis of a DNA molecule encoding the antibody and the DNA molecule expresses an antibody protein, or an amino acid sequence specifying the antibody, wherein the DNA or amino acid sequence has been obtained using synthetic DNA or amino acid sequence technology which is available and well known in the art.

As used herein, the phrase “antibody against soluble CD163” refers to an antibody that binds to at least one extracellular epitope present on soluble CD163. In one embodiment, the “antibody does not bind macrophage-associated CD163,” that is the antibody binds to soluble CD163 but does not substantially bind CD163 present in the membrane of a macrophage/monocyte. In other embodiments, less than 50% of the antibody added binds CD163; less than 40%, less than 30%, less than 20%, less than 10%, less than 5%, or even less than 1% of the antibody binds to membrane-bound CD163. Alternatively, when cellular sources of CD163 (e.g., macrophages/monocytes) are removed from the sample obtained from an individual, a CD163 antibody that recognizes both membrane-associated and soluble CD163 can be used. In general, it is preferred that measurement of sCD163 is performed in the absence of cross-contamination with membrane-associated CD163, and this can be achieved by either using an antibody that does not substantially bind membrane-associated CD163 or using a sample that does not substantially contain membrane-associated CD163 but retains sCD163. A sample that does not substantially contain membrane-associated CD163 but retains sCD163 is referred to herein as a “monocyte-depleted biological sample.” Antibodies against CD163 and soluble CD163 are commercially available. In some embodiments, the antibody is an antibody described in Sulahian T H et al. (Development of an ELISA to measure soluble CD163 in biological fluids. J Immunol Meth. 2001; 252:25-31). In some embodiments, the antibody is Anti-CD163 monoclonal antibodies (R20 and D7, IgG1) as described in Matsushita et al. (Clin Exp Immunol. 2002 October; 130(1): 156-161). In some embodiment, two antibodies are used for the detection as described in U.S. Pat. No. 7,144,710. Thus, the antibody can comprise a capture antibody and a detection antibody both specific for soluble CD163. In some embodiments, the sCD163 capture antibody is MAC2-158 or MAC2-48 and the sCD163 detection antibody is RM3/1 as described in U.S. Pat. No. 7,144,710. In some aspects of all the embodiments of the invention, the antibody is a monoclonal antibody. In some aspects of all the embodiments of the invention, the antibody is clone CD163, MAC2-158, Isotype IgGlk, (Trillium Diagnostics, LLC). The antibody may be labeled or unlabeled. Labeling of antibodies is well known to one skilled in the art. The assays one can use the antibodies include at least flow cytometry, ELISA, Western blot, and fluorescent microscopy.

As used herein the phrase “risk of onset of a macrophage-mediated disease” refers to an increased level of sCD163 of at least 5-10% in an HIV+ individual as compared to a standard. Alternatively, “risk of onset” refers to an increased level of sCD163 in an HIV+ individual as measured at a second time point compared to the level of sCD163 measured at a first time point. In other embodiments, the level of sCD163 (compared to either a standard or the level of sCD163 at an at least second time point) is increased by at least 30%, at least 40%, at least 50%, at least 60%, at least 70%, at least 80%, at least 90%, at least 95%, at least 99%, at least 1-fold, at least 2-fold, at least 5-fold, at least 10-fold, at least 50-fold, at least 100-fold, at least 1000-fold or more. The risk of onset of a macrophage-mediated disease can also take into account other factors such as, family history of macrophage-mediated disease, previous medical history of the individual, antiretroviral therapy (e.g., successful vs. unsuccessful), diagnosis of HIV+, viral load, level of T-cell lymphocytes, and stage of disease when diagnosed (e.g., acute vs. chronic). Such methods for determining risk of disease onset in an individual is well known to those of skill in the art and is routinely performed in a clinical setting.

As used herein, the phrase “at a subsequent time period” refers to measurement of sCD163 levels at two or more time points (e.g., 3, 4, 5, 6, 7, 8, 9, 10, 11, 12, 13, 14, 15, 16, 17, 18, 19, 20, 25, 50, or more). It is contemplated herein that an individual can be continually monitored for levels of sCD163 to predict the risk of HIV in an individual who has not undergone seroconversion, or to predict the risk of complications of HIV developing in the individual infected with HIV. For example, an individual can be monitored for sCD163 levels at a frequency of e.g., every 6 h, every 12 h, every 24 h, every 48 h, every 72 h, every 4 days, every 5 days, every 6 days, every week, every 2 weeks, every 3 weeks, every 4 weeks, every month, every 6 weeks, every 2 months, every 3 months, every 4 months, every 5 months, every 6 months, every 7 months, every 8 months, every 9 months, every 10 months, every 11 months, every year, every 2 years, every 5 years, every 10 years, or even every 20 years. One of skill in the art can also choose to obtain a sample at a discrete time point based on the apparent health of the individual, suspected lack of success using anti-retroviral therapies, history of macrophage-mediated disease or when one of skill in the art suspects that a macrophage-mediated disease is developing based on symptoms.

As used herein, the phrase “determining or monitoring the effectiveness of a treatment” refers to a method of measuring the level of sCD163 before and after administering a treatment to an individual, and determining if the level of sCD163 has gone up (e.g., worsening of disease, poor efficacy of treatment), down (e.g., improvement of disease status, good efficacy of treatment), or stayed the same (e.g., no appreciable efficacy of treatment).

As used herein, the term “test model” refers to a subject infected by HIV or a related virus (e.g., SIV). In one embodiment, the subject is a human, such as in an approved clinical trial. In another embodiment, the subject is a non-human primate (e.g., a simian, a gorilla, a chimpanzee, an orangutan, a baboon, a New World monkey, a gibbon, a great ape, a tamarin, a marmoset, a night monkey, an owl monkey, etc.). A test model can be used at any stage of disease (e.g., acute HIV+ infection, early infection (i.e. within one year of infection), chronic HIV+ infection, HIV+ undergoing classical ART therapy, HIV+ with early symptoms of AIDS, and AIDS).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### BRIEF DESCRIPTION OF THE DRAWINGS

FIGS. 1A-1D show that sCD163 is significantly increased in plasma during chronic and early HIV infection and decreased after ART. Plasma from 30 chronically and 14 early HIV-infected subjects was examined at 2 time points: 1) no current ART (Chronic HIV+, Pre-ART) (Early HIV+, Pre-ART) and 2) after 3 months of ART (Chronic HIV+, 3mos ART) (Early HIV+, 3mos ART). Samples were compared to 29 age-matched HIV-seronegative (HIV-). Plasma was examined for levels of sCD163 (FIG. 1A), IL-10 (FIG. 1B), sCD14 (FIG. 1C) and LPS (FIG. 1D). (FIG. 1A) Plasma sCD163 was elevated pre-ART in both chronic and early HIV infected individuals. After 3 months of ART sCD163 decreased in both chronic and early patients, however, the levels only returned to uninfected levels in early HIV subjects. The lines show the means and standard error of the mean. An ANOVA was used to test for variance between groups. If the ANOVA was significant (p<0.05) then this was followed by post-hoc t-tests. For comparisons between Chronic HIV+Pre-ART versus Chronic HIV+3mos ART and between Early HIV+Pre-ART versus Early HIV+3mos ART matched t-tests were used. Samples from three uninfected, three early HIV-infected Pre-ART and eight early 3mos-ART samples were below the detection of for the LAL assay.

FIGS. 2A-2B show that sCD163 in plasma was significantly decreased after ART in matched, chronic HIV-infected individuals. In order to determine the effect of successful ART on soluble plasma factors, matched samples were examined prior to (Chronic HIV+, Pre-ART) and after 3 months of ART (Chronic HIV+, 3mos ART). Soluble factors examined were sCD163, IL-10, sCD14, LPS, IL-6 and osteopontin (OPN). All P values shown were calculated using a two-tailed paired t-test. Results for sCD163 are shown. A multiple comparison adjustment was used (/n) where a significant P<0.02. P=9×10⁻⁸ in FIG. 2A, and **P=0.0007, in FIG. 2B indicate a statistically significant result.

FIGS. 3A-3D show that the percent change in plasma virus and absolute number of CD4+ T lymphocytes correlated with change in plasma sCD163 levels after ART. (FIG. 3A, FIG. 3B) In order to directly measure the effect of ART on virological parameters examined in chronic HIV-infected individuals, the percent change was calculated (((Pre ART−3mos ART)/Pre ART)*100%), where a negative percentage represents a decrease and a positive percentage represents an increase after 3 months of ART. The percent change in sCD163 was correlated to the percent change in plasma virus (FIG. 3A), absolute number of CD4+ T lymphocytes (B). (FIG. 3C, FIG. 3D). The absolute number of monocytes correlated to sCD163 levels in plasma from chronically HIV-infected subjects prior to ART (FIG. 3C) but not after 3 months of ART (FIG. 3D). r=Spearman's correlation coefficient, P value of ≦0.05 is significant.

FIGS. 4A-4D show that The percentage of CD14+ CD16+ monocytes and CD8+ HLA-DR+CD38+ T lymphocytes correlates with sCD163. Peripheral blood monocytes (PBMC) from 8 chronic (closed circles) HIV patients Pre-ART and 9 patients after 3mos ART (2 sets of samples were from matched patients) and PBMC from all 14 early (open circles) HIV-infected individuals Pre-ART and with 3mos ART were examined. Flow cytometry analysis was used to determine the percentage of CD14+ CD16+ monocytes (FIG. 4A, FIG. 4B), the median fluorescence intensity (MFI) of CD163 on CD14+ CD16+ monocytes (FIG. 4C, FIG. 4D) and the percentage of activated CD8+ HLA-DR+CD38+ T lymphocytes (FIG. 4E, FIG. 4F) in all PBMC samples. (FIG. 4A, FIG. 4C, FIG. 4E) Non-parametric Mann-Whitney t tests were used where a P value of ≦0.05 is significant. For the early HIV+ samples, paired t tests were used since all samples were matched Pre-ART and 3mos ART. (FIG. 4B, FIG. 4D, FIG. 4F) The correlations were performed with both early HIV positive individuals (open circles) and chronic HIV infected individuals (closed circles). (FIG. 4B) The percentage of CD14+ CD16+ monocytes significantly correlated to plasma sCD163. (FIG. 4D) The expression level of CD163 on CD14+ CD16+ monocytes inversely correlated to plasma sCD163. (FIG. 4F) The percentage of activated CD8+ HLA-DR+CD38+ T lymphocytes significantly correlated to plasma sCD163 in early HIV-infected patients. The r and P shown are for were the overall correlation including both early and chronic patients. r=Spearman's correlation coefficient, P value of ≦0.05 is significant.

FIGS. 5A-5I show sCD163, plasma viral load and CD8+ T lymphocytes monitored over the first year after seroconversion in nine early HIV-infected individuals. Plasma was examined for sCD163 (circle “O” left axis), plasma viral load (triangle “Δ” right axis) and absolute number of CD8+ T lymphocytes (square “□” left axis). Samples were plotted over the first year of infection from seroconversion (seroconversion=day 0 from +ELISA). CD4+ T lymphocytes were also examined, but did not parallel sCD163 or plasma viral load. Shaded gray area=currently on ART, white area=no ART.

FIGS. 6A-6D show that plasma virus and CD8+ T lymphocytes, but not CD4+ T lymphocytes parallels sCD163 levels in plasma, even during ART interruption. Plasma was examined for sCD163 (circle “O” left axis), plasma viral load (triangle “Δ” right axis), absolute number of CD8+ T lymphocytes (square “□” left axis) and absolute number of CD4+ T lymphocytes (upside down triangle “∇” left axis). Shaded gray area=currently on ART, white area=no ART. Shown are two representative early HIV-infected individuals out of four examined during periods of ART interruption and re-initiation. sCD163 increased, paralleling plasma viral load and absolute numbers of CD8+ T lymphocytes during interrupted therapy and decreased upon re-initiation of ART. CD4+ T lymphocytes were not affected by therapy interruption.

FIGS. 7A-7B show that plasma sCD163 positively correlates with percentage and absolute number of CD14+ CD16+ monocytes in SIV-infected rhesus macaques. Plasma sCD163 levels in six SIV-infected rhesus macaques correlated to the percentage of CD14+ CD16+ monocytes (A) and the absolute number of CD 1 4+ CD 16+ monocytes (B).

FIG. 8 is a block diagram depicting an exemplary system for use with the diagnostic methods described herein.

FIG. 9 is a block diagram depicting exemplary instructions encoded on a computer readable storage medium for use with the systems described herein.

FIGS. 10A-10C show a series of graphs showing correlation of sCD163 with coronary plaques in HIV+ and serotype negative individuals.

FIG. 11 shows sCD163 levels in HIV-patients with or without detectable HIV RNA levels vs. HIV-seronegative controls Results are mean±SEM.

FIGS. 12A-12C show a comparison of sCD163, LPS and prevalence of coronary plaque in HIV-seronegative controls vs. HIV patients on ART with viral suppression. Results are mean±SD.

FIG. 13 shows the global dementia score (GDS) related to sCD163 plasma levels in HIV+ patients with normal global dementia score and HIV+ patients with global dementia score indicating impaired mental functionality.

### DETAILED DESCRIPTION

The assays and methods provided herein are based, in part, on the discovery that levels of soluble CD163 (sCD163) are increased in HIV+ individuals compared to HIV-serotype negative individuals. Further, the inventors have discovered that treatment of HIV+ individuals with antiretroviral drugs (ART) reduces the level of sCD163 compared to HIV+ individuals who are not treated with ART. Thus, the inventors have discovered that the levels of sCD163 correlate with HIV-disease activity, assessed by measuring e.g., HIV viral replication detected in plasma or cerebral spinal fluid. Individuals infected with HIV and in whom the viral infection is not successfully controlled using ART are at a higher risk of developing HIV-related disorders, particularly macrophage-mediated diseases. Thus, based on the novel findings regarding the soluble CD163 levels, this application provides methods and assays for determining the risk of an HIV+ individual for developing a macrophage-mediated disease. Also provided herein are assays and methods for monitoring efficacy of a treatment for a macrophage-mediated disease, and methods for screening for agents to treat a macrophage-mediated disease in an HIV+ individual. The invention further provides assays to diagnose heart disease in an individual using sCD163 as an early indicator. In some embodiments, the individual is infected with HIV.

Monocytes/macrophages constitute an important cellular component of immune responses against viruses. They serve as antigen presenting cells and also produce inflammatory mediators that activate additional cellular components of innate immunity and thus provide a bridge to the adaptive immune responses (Christensen, J. E., and Thomsen, A. R. 2009. APMIS 117:338-355). Macrophage activation is thought to play a pivotal role in pathogenesis of human immunodeficiency virus (HIV) infection, where expansion within blood of specific subsets of monocyte/macrophages is observed and may, in part drive pathogenesis (Hasegawa, A., et al. 2009. Blood; Williams, K. C., and Hickey, W. F. 2002. Annu Rev Neurosci 25:537-562).

HIV-mediated destruction of gut mucosal epithelial CD4+ T cells and translocation of microbial products, known as pathogen associated molecular patterns (PAMPs), into the systemic circulation occurs early in HIV disease (Veazey, R. S., and Lackner, A. A. 2004. J Exp Med 200:697-700; Brenchley, J. M., et al., 2006. Nat Med 12:1365-1371; Ancuta, P., et al. 2008. PLoS ONE 3:e2516). PAMPs, such as lipopolysaccharide (LPS), are thought to activate innate immune responses and monocyte/macrophages via toll-like receptors (TLRs) (Akira, S., et al., 2006. Cell 124:783-801; Ishii, K. J., et al., 2008. Cell Host Microbe 3:352-363; Pichlmair, A., and Reis e Sousa, C. 2007. Immunity 27:370-383). Uncontrolled hyper-reactivity of such innate immune pathways can result in sepsis, chronic immune activation and possibly immune exhaustion (Zhao, J., et al., 2009. Trends Immunol 30:8-12; Sachdeva, M., et al., 2010 J Acquir Immune Defic Syndr). Recent studies have shown that microbial translocation may be responsible for systemic immune activation during AIDS, although monocyte/macrophages are likely sensitized to LPS stimulation over time (Brenchley, J. M., et al., 2006. Nat Med 12:1365-1371; Noursadeghi, M., et al., 2006. Lancet Infect Dis 6:794-804).

The hemoglobin scavenger receptor CD163 is expressed exclusively by monocyte/macrophages and selectively by macrophages considered of the M2 phenotype (Weaver, L. K., et al., 2006. J Leukoc Biol 80:26-35; Hintz, K. A., et al., 2002. J Leukoc Biol 72:711-717; Moller, H. J., et al., 2002. Blood 99:378-380; Backe, E., et al., 1991. J Clin Pathol 44:936-945). Thus, surface CD163 is considered to be a marker of alternatively activated (anti-inflammatory) macrophages (Moestrup, S. K., and Moller, H. J. 2004. Ann Med 36:347-354). Extracellular TLR activation leads to shedding of cell surface CD163, giving rise to a soluble form (sCD163) that contains the entire extracellular domain. Since surface CD163 on macrophages has been shown to function as an innate immune receptor for bacteria (Fabriek, B. O., et al., 2009. Blood 113:887-892), its shedding may be a mechanism to decrease acute and severe monocyte activation and inflammation (Akira, S., et al., 2006. Cell 124:783-801). LPS, in a dose-dependent manner (picogram range), at levels similar to those seen in HIV-infected individuals (Brenchley, J. M., et al. 2006. Nat Med 12:1365-1371; Ancuta, P., et al. 2008. PLoS ONE 3:e2516) acutely stimulates shedding within 1 hour, followed by increased expression on the surface of monocyte/macrophages 24 to 72 hours later (Weaver, L. K., et al., 2006. J Leukoc Biol 80:26-35). CD163 expression on monocytes has been shown to inversely correlate with sCD163 levels both in vitro and in vivo (Weaver, L. K., et al., 2006. J Leukoc Biol 80:26-35, Davis, B. H., and Zarev, P. V. 2005. Cytometry B Clin Cytom 63:16-22; Weaver, L. K., et al., 2007. J Leukoc Biol 81:663-671). In vivo shedding of sCD163 results from LPS ligation of TLRs (Weaver, L. K., et al., 2006. J Leukoc Biol 80:26-35, Hintz, K. A., et al., 2002. J Leukoc Biol 72:711-717), Fc (gamma) receptor cross-linking (Sulahian, T. H., et al., 2004. J Leukoc Biol 76:271-277), or oxidative stress mediators present during inflammation (Timmermann, M., and Hogger, P. 2005. Free Radic Biol Med 39:98-107) that are cleaved by metalloproteinases (Timmermann, M., and Hogger, P. 2005. Free Radic Biol Med 39:98-107; Droste, A., 1999. Biochem BiophysRes Commun 256:110-113). In addition, levels of sCD163 in plasma are elevated in association with macrophage-directed diseases, including sepsis (Moller, H. J., 2002. Blood 99:378-380) and Gaucher's disease (Moller, H. J., et al., 2004. Eur J Haematol 72:135-139), the latter of which is characterized by macrophage accumulation in the liver and spleen (Moller, H. J., et al., 2004. Eur J Haematol 72:135-139).

Bacterial binding to surface CD163 on macrophages triggers cytokine production (Fabriek, B. O., et al., 2009. Blood 113:887-892). Monocyte subsets CD14+ and CD14+ CD16+ produce sCD163, where the CD14+ CD16+ population has the highest expression and presumably shedding (Buechler, C., et al., 2000. J Leukoc Biol 67:97-103) of sCD163. Further, an increased percentage and absolute number of CD14+ CD16+ monocytes with HIV infection correlate with histopathology and HIV-associated dementia (Ancuta, P., et al., PLoS One. 2008 Jun. 25; 3(6):e2516; Pulliam, L., et al., 1997. Lancet 349:692-695; Kim, W. K., et al., 2009. J Leukoc Biol; Burdo, T. H., et al., 2010. PLoS Pathog 6:e1000842.6, 26-28).

The levels of sCD163 in people living with HIV, the associations of sCD163 with disease characteristics, and the clinical utility of sCD163 measurement in HIV disease management are described herein. Although CD163 on macrophages has been described for use in diagnosis of HIV, research regarding sCD163 shows that levels of sCD163 are not correlated with macrophage number under all conditions. However, we now provides evidence to demonstrate that sCD163 correlates with HIV disease status in individuals infected with HIV.

Recently, it was demonstrated that monocyte expansion from bone marrow to blood, measured by BrdU expression, during simian immunodeficiency virus (SIV) infection correlated with the rate of progression to AIDS (Hasegawa, A., et al., 2009. Blood;. W. K., et al., 2009. J Leukoc Biol; Burdo, T. H., et al., 2010. PLoS Pathog 6:e1000842.6, 26-28) and severity of SIV encephalitis (SIVE) (W. K., et al., 2009. J Leukoc Biol; Burdo, T. H., et al., 2010. PLoS Pathog 6:e1000842.6, 26-28).

Monocyte/macrophage expansion, more so than plasma virus load and changes in CD4+ T cell numbers, correlated with the development of AIDS (Hasegawa, A., et al., 2009. Blood;). The stimulus for such expansion with infection is not defined; however, the best correlate to the magnitude of BrdU uptake into monocytes was sCD163 in plasma, more so than LPS and CCL2/monocyte chemoattractant protein 1 (MCP-1) levels (Burdo, T. H., et al., 2010. PLoS Pathog 6:e1000842.6, 26-28). The correlation between sCD163 in plasma, that is uniquely made by monocytes, with the magnitude of monocyte expansion, underscores the importance of innate immune activation in AIDS. Furthermore, absolute numbers of CD163 expressing monocytes are elevated during SIV and HIV infection (Kim, W. K., et al., 2006. Am J Pathol 168:822-834; Fischer-Smith, T., et al., 2008. J Neurovirol 14:318-326.29, 30).

Diagnostic Methods in Individuals not Known to be Infected with HIV

In one embodiment, assays and methods described herein relate to determining risk of HIV infection in an individual that has not yet undergone seroconversion (e.g., does not have detectable levels of HIV antibodies in a biological sample). For example, an increase in sCD163 in an individual indicates that an individual has been infected with HIV. Such methods can allow for an earlier diagnosis of HIV, prior to the appearance of HIV antibodies in the individual. The current detection methods solely rely on diagnosis after seroconversion and thus, the present method provides an earlier diagnostic tool compared to the methods currently available in the art. Accordingly, the invention provides a method of detecting HIV infection comprising contacting a biological sample, such as blood, plasma or cerebrospinal fluid, from an individual with one or more antibodies that detect soluble CD163 in the biological sample and comparing the amount of soluble CD163 in the biological sample to a reference, wherein an increase of at least 5-10% in the amount of soluble CD163 in the biological sample compared to the reference is indicative of the increased risk that the individual is infected with HIV. In some embodiments, the method further comprises administering to the individual at risk of being infected with HIV and antiretroviral therapy. In some embodiments, the individual is an individual suspected to have been in contact with an HIV positive individual or biological fluid, such as blood or sperm, and not having other inflammatory symptoms.

Also provided herein are assays and methods for diagnosing heart disease (e.g., coronary atherosclerosis or cardiomyopathy) in individuals not infected with HIV. In such assays and methods, an increase in sCD163 of at least 10% in a biological sample is indicative of an increased risk or the presence of a heart disease or cardiovascular disease such as coronary atherosclerosis, e.g., non-calcified coronary atherosclerosis. In some embodiments, the individual from whom the biological sample is obtained has not, at least knowingly, been in contact with an HIV positive individual or biological fluid, such as blood or sperm.

### Diagnosis of HIV and AIDS

In some embodiments, the assays and methods described herein are directed at assessing risk or monitoring treatment of a macrophage-mediated disease in an individual infected with HIV. Thus, the individuals and subjects described herein will have been first diagnosed as being HIV infected (HIV positive). Methods for diagnosing HIV are well known in the art and are described briefly herein.

The most common screening test used to detect antibodies to HIV is an enzyme immunoassay (EIA) test performed using blood drawn from a vein. A positive (reactive) EIA is used with a follow-up (confirmatory) test such as a Western blot to make a positive diagnosis. There are EIA tests that can use other body fluids to detect antibodies to HIV. For example, an oral fluid test can be used, which uses oral fluid (not saliva) that is collected from the mouth using a special collection device. This is an EIA antibody test similar to the standard blood EIA test. A follow-up confirmatory Western blot uses the same oral fluid sample. A urine test can also be performed instead of using blood as the biological sample; however the sensitivity and accuracy are not as high as that of the blood and oral fluid tests. The urine test is based on an EIA antibody test similar to blood EIA tests and requires a follow-up confirmatory Western blot using the same urine sample.

Rapid tests for diagnosing HIV are also available. A rapid test is a screening test that produces very quick results, in approximately 20 minutes. Rapid tests use blood from a vein or from a finger stick, or oral fluid, to look for the presence of antibodies to HIV. As is true for all screening tests, a reactive rapid HIV test result must be confirmed with a follow-up confirmatory test before a final diagnosis of infection can be made. These tests have similar accuracy rates as traditional EIA screening tests.

An individual can also perform a home test for HIV. Consumer-controlled test kits (popularly known as “home testing kits”) were first licensed in 1997. Although home HIV tests are sometimes advertised through the Internet, currently only the Home Access HIV-1 Test System is approved by the Food and Drug Administration. (The accuracy of other home test kits cannot be verified). The Home Access HIV-1 Test System can be found at most local drug stores. It is not a true home test, but a home collection kit. The testing procedure involves pricking a finger with a special device, placing drops of blood on a specially treated card, and then mailing the card in to be tested at a licensed laboratory. Customers are given an identification number to use when phoning in for the results. Callers may speak to a counselor before taking the test, while waiting for the test result, and when the results are given. All individuals receiving a positive test result are provided referrals for a follow-up confirmatory test, as well as information and resources on treatment and support services.

Alternatively, RNA tests can be used to detect viral genetic material and can be used in screening the blood supply and for detection of rare very early infection cases when antibody tests are unable to detect antibodies to HIV.

For diagnosis of an individual with HIV, such diagnosis is preferably performed using a licensed ELISA kit and confirmed by Western blot. Additional HIV infection confirmation tests can include, but are not limited to, HIV culture, HIV antigen, plasma HIV RNA and other secondary antibody tests other than ELISA. Tests for diagnosis of various categories of disease associated with HIV infection are well known in the art and are commercially available.

HIV infection can lead to symptoms of Acquired Immune Deficiency Syndrome (AIDS) in an individual or subject. The Centers for Disease Control and Prevention classify a patient as having AIDS when HIV infection is confirmed via an accepted testing method, the CD4 positive cell count is less than 200 cells per cubic milliliter, or when CD4 positive cells are less than 14 percent of the total lymphocyte population, and one of the opportunistic infections listed below is present. The list of opportunistic infections includes: candidiasis in the bronchi, trachea, or lungs, esophogeal candidiasis, invasive cervical cancer, disseminated or extrapulmonary coccidioidomycosis, extrapulmonary cryptococcosis, chronic intestinal cryptosporidiosis (greater than one month's duration), cytomegalovirus disease in a location other than the liver, spleen, or nodes, cytomegalovirus retinitis with loss of vision, HIV-related encephalopathy, herpes simplex with chronic ulcer(s) greater than one month's duration or bronchitis, pneumonitis, or esophagitis, disseminated or extrapulmonary histoplasmosis, chronic intestinal isosporiasis (greater than one month's duration), Kaposi's sarcoma, Burkitt's lymphoma, (or equivalent term), immunoblastic lymphoma (or equivalent term), primary lymphoma of the brain, disseminated or extrapulmonary Mycobacterium avium complex or M. kansasii, pulmonary or extrapulmonary Mycobacterium tuberculosis at any site, disseminated or extrapulmonary Mycobacterium of other species or unidentified species, pneumocystis carinii pneumonia, recurrent pneumonia, progressive multifocal leukoencephalopathy, recurrent Salmonella septicemia, toxoplasmosis of brain, and wasting syndrome due to HIV.

### Macrophage-Mediated Disease

The assays and methods provided herein can be used to assess risk of a subject, e.g., a human individual, for developing a macrophage-mediated disease and methods to monitor efficacy of a treatment for a macrophage-mediated disease administered to an HIV infected individual. The assays and methods are typically in vitro assays and methods. Macrophage-mediated diseases commonly associated with HIV include, for example, AIDS-related dementia, peripheral neuropathy and HIV-associated heart disease. However, the methods described herein can be used to assess risk or monitor treatment of any macrophage-mediated disease in an HIV infected individual. A macrophage-mediated disease is a disease associated with an elevated or abnormal level of macrophage proliferation or activation as compared to a control sample.

In one embodiment, the methods described herein can be used to assess risk of disease development or monitor treatment efficacy of an autoimmune disease including, for example, AIDS-associated dementia, Alzheimer's disease, amyotrophic lateral sclerosis, AIDS lymphoma, follicular lymphoma, mycoses fungoides, age-related macular degeneration (ARMD), atherosclerosis, kidney disease (such as focal segmental glomerulosclerosis and membrane proliferative glomerulonephropathy), AIDS-associated diarrhea, diabetes mellitus, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

This list by no means intends to list all of the macrophage-mediated diseases but provides a list of several clinically relevant diseases. One of skill in the art would know if a disease is mediated by abnormal macrophage function or number and then apply the methods described herein to assess risk or monitor treatment efficacy in individuals infected with HIV.

Also contemplated herein is a treatment of macrophage mediated diseases in HIV infected individuals. Any treatment known in the art can be used to treat an individual infected with HIV and further affected with a macrophage-mediated disorder. For example, mitoguazone (MGBG) can be used to reduce the HIV viral load of an individual and also treat a macrophage-mediated disease (see e.g., U.S. Patent Application No. US2007/0078187, which is herein incorporated by reference in its entirety). Mitoguazone is also known in the art as 1,1′[ methylethanediylidene]dinitrilo-)diguanidine, methylglyoxal bis(guanylhydrazone), and methyl-GAG. MGBG can be used as a free base or salts thereof. In one embodiment, MGBG is supplied as a dihydrochloride. The application provides a possibility for early intervention by also providing an assay for diagnosis combined with treatment if an early disgnosis is established, i.e., if sCD163 levels are increased in the tested sample.

In some embodiments, when an increase in the soluble CD163 level is detected, the individual is further prescribed or administered a treatment or an additional treatment or a modified treatment from the treatment the individual is already on as a treatment or prophylactic to slow down or inhibit development of the macrophage-associated disease.

### Biological Samples

A biological sample can be obtained from any organ or tissue in the individual to be tested, provided that the biological sample comprises macrophages/monocytes. Such samples can be further processed to remove the macrophages/monocytes and other cells, thereby producing a monocyte-depleted sample. For example, plasma and serum can be isolated from a whole blood sample by e.g., treating the whole blood sample with an anticoagulant such as heparin and centrifuging the sample until the cells sediment to permit plasma to be removed from the upper aqueous layer. Methods for removing cells from a blood or other biological sample are well known in the art and can include e.g., centrifugation, ultrafiltration, immune selection, or sedimentation etc.

Proteins and nucleic acids can be detected from a biological sample or a sample that has been treated as described above or as known to those of skill in the art.

Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a sperm sample, a stool sample, a biopsy sample, a needle aspiration biopsy sample, a swab sample, a mouthwash sample, a cancer sample, a tumor sample, a tissue sample, a cell sample, a cell lysate sample, a crude cell lysate sample, a production sample, a protein preparation sample, or a combination of such samples. For the methods described herein, one typically uses a biological sample that is obtained from whole blood, plasma, cerebral spinal fluid, platelets, serum, saliva, sputum, and/or urine.

### Antibody Compositions

sCD163 protein from the biological sample to be analyzed can be detected or isolated using techniques which are well known to one of skill in the art, including but not limited to Western blot analysis, (i.e.), immunoblotting, ELISA, immunoprecipitation, lateral flow immunoassay, radioimmunoassay, etc. Antibodies directed against a sCD163 peptide can be applied for disease diagnostics and prognostics. Such diagnostic methods and assays can be used to detect levels of sCD163 shed from the macrophage/monocytic cells. In most instances, it will be the amount of sCD163 that is of primary interest. Antibodies to be used for protein analysis are widely available through commercial sources including AbCam (Cambridge, Mass.), New England Biolabs (Ipswich, Mass.), Santa Cruz Biotechnologies (Santa Cruz, Calif.), AbD Serotec, and Cell Signaling (Danvers, Mass.), among others. Antibodies can also be raised against a polypeptide or portion of a polypeptide, such as the extracellular domain of CD163 by methods known to those skilled in the art. Such methods are readily available and well known to those of skill in the art.

In some embodiments, the antibody is anti-CD163 monoclonal antibodies R20 and D7, IgG1 as described in Matsushita et al. (Clin Exp Immunol. 2002 October; 130(1): 156-161). In some aspects of all the embodiments of the invention, two antibodies are used for the detection as described in U.S. Pat. No. 7,144,710. Thus, the antibody can comprise a capture antibody and a detection antibody both specific for soluble CD163. In some aspects of all the embodiments of the invention, the sCD163 capture antibody is MAC2-158 or MAC2-48 and the sCD163 detection antibody is RM3/1 as described in U.S. Pat. No. 7,144,710. In some aspects of all the embodiments of the invention, the antibody is a monoclonal antibody. In some aspects of all the embodiments of the invention, the antibody is clone CD163, MAC2-158, Isotype IgGlk, (Trillium Diagnostics, LLC). The antibody may be labeled or unlabeled. Labeling of antibodies is well known to one skilled in the art. The assays one can use the antibodies include at least flow cytometry, ELISA, Western blot, and fluorescent microscopy.

The 1009 amino acid (aa) extracellular domain of CD163 contains nine scavenger receptor cysteine-rich (SRCR) domains (Law, S. K. A. et al. (1993) Eur. J. Immunol. 23:2320). The third SRCR domain is crucial for calcium-dependent binding of hemoglobin/haptoglobin complexes (Madsen, M. et al. (2004) J. Biol. Chem. 279:51561.). Four isoforms vary in both intracellular and extracellular regions (Law, S. K. A. et al. (1993) Eur. J. Immunol. 23:2320; Nielsen, M. J. et al. (2006) J. Leukoc. Biol. 79:837.). The C-terminal 42 amino acids of the 84 amino acid cytoplasmic domain are substituted with 48 alternate amino acids in isoform 2. The same region is substituted by six alternate amino acid in isoforms 3 and 4. Isoform 4 also has a 34 amino acid insert between SRCR domains 5 and 6. While all isoforms are expressed, isoform 3 is the most abundant isoform and it is most expressed on the cell surface, and most active in endocytosis (Nielsen, M. J. et al. (2006) J. Leukoc. Biol. 79:837). Without wishing to be bound by a theory, an approximately 130 kDa soluble form of human CD163 (sCD163) contains virtually all of the extracellular domain. It shares approximately 75% amino acid sequence identity with mouse and rat sCD163 (Moller, H. J. et al. (2002) Blood 99:378; Droste, A. et al. (1999) Biochem. Biophys. Res. Comm. 256:110). It is released from the cell surface by proteolysis after oxidative stress or inflammatory stimuli, including bacterial endotoxins and activation of the Toll-like receptors TLR2 or TLR5 Droste, A. et al. (1999) Biochem. Biophys. Res. Comm. 256:110; Hintz, K. A. et al. (2002) J. Leukoc. Biol. 72:711; Weaver, L. K. et al. (2006) J. Leukoc. Biol. 80:26; Timmerman, M. and P. Hogger (2005) Free Radic. Biol. Med. 39:98). In some aspects of all the embodiments of the invention, the antibodies are specific to the extracellular domain of isoform 3. In some aspects of all the embodiments of the invention, the antibodies are specific to the extracellular domain of isoform 4. In some aspects of all the embodiments of the invention, the antibody is designed to recognize all isoforms in their soluble, shed form.

Antibodies against the soluble CD163 can be raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. While both polyclonal and monoclonal antibodies can be used in the methods described herein, it is preferred that a monoclonal antibody is used where conditions require increased specificity for sCD163. Antibody manufacture methods are described herein, for example, in Harlow et al., 1988. In one embodiment described herein, an ELISA or a Western Blot detects the presence, or absence of a polypeptide as well as the extent of polypeptide level or concentration. For a detailed explanation of methods for carrying out Western blot analysis, see Sambrook et al., 1989, at Chapter 18. Protein detection and isolation methods that can be employed in the methods described herein are also described in Harlow and Lane, 1988. The antibodies that recognize the factor may be any antibody variant, antibody derivative, bispecific molecule, human antibody, humanized antibody, monoclonal antibody, human monoclonal, and variants and antigen-binding fragments thereof. Conventional methods for immunohistochemistry are described in Harlow and Lane, 1988 and Ausbel et al, 1987. Specificity of the antibody can be screened using routine methods. Antibodies that recognize both macrophage-associated CD163 and soluble CD163 are discarded from the methods and assays of the invention as non-specific to the soluble CD163.

### Standards/References

The terms “standard” and “reference” are used herein interchangeably. A standard or a reference can permit one of skill in the art to determine the amount of sCD163 or the relative increase/decrease of sCD163 in a biological sample. A standard/reference serves as a reference level for comparison, such that samples can be normalized to an appropriate standard in order to infer the presence, absence or extent of a macrophage-mediated disease in an HIV+ individual. In one embodiment, a standard/reference is obtained from the same individual as that being tested at an earlier time point. Thus, a sample obtained from a patient is compared to a previously obtained sample, which acts as a reference. This type of standard is generally the most accurate for diagnostic and prognostic purposes, since a majority of other markers will remain relatively similar from sample to sample in one individual. The standard/reference is ideally obtained prior to the suspected onset of a macrophage-mediated disease, when the levels of sCD163 is at a baseline level for that individual at the time of testing. However, a standard/reference can be obtained from an individual after the onset of a macrophage-mediated disease as it can still provide information about improvement of symptoms or regression of the disease following treatment. For example, in some cases an increase in the amount of sCD163 in a biological sample from an HIV+ individual can detect an increase in the risk of developing a macrophage-mediated disease or a failure of a treatment to slow disease progress, while a decrease in amount of sCD163 in a biological sample from an individual can indicate a regression of the disease or a decrease in risk of developing a macrophage-mediated disease.

A standard can also be obtained from another individual or a plurality of individuals, wherein a standard represents an average level of sCD163 among a population of HIV+ individuals with or without HIV-associated/macrophage-associated disease(s) or a population of individuals not infected with HIV. Thus, the level of sCD163 in a standard obtained in this manner is representative of an average level of this factor in the given population, such as a general population of individuals infected by HIV. An individual sample is compared to this population standard by comparing levels of sCD163 from a biological sample relative to the standard. Generally, an increase in the amount of sCD163 will indicate an increased risk of a HIV-related complications such as a macrophage-mediated disease, or a progression in HIV disease activity, while a decrease in the sCD163 amount will indicate a reduced risk of macrophage-mediated disease as well as a regression in HIV disease activity.

For the purposes of the methods and assays of the invention, a reference may be a result from a parallel sample analyzed with the test sample or a reference value or a range of reference values, e.g., in a database. Computer-implemented software can be used to produce and/or perform the comparison between the sample and the reference. An output of the computerized comparison can be in the form of a screen or a printout or a generated message to be sent, e.g., via e-mail or text message.

It should be noted that there can be variability among individuals in a population, such that some individuals will have high or very high levels of sCD163, while other individuals have very low or very levels of sCD163. However, one skilled in the art can make logical inferences on an individual basis regarding the determination of risk as described herein.

A standard/reference or series of standards/references can also be synthesized. A known amount of sCD163 (or a series of known amounts) can be prepared within the typical expression range for a factor that is observed in a general HIV+ population. This method has an advantage of being able to compare the extent of disease in two individuals in a mixed population. This method can also be useful for individuals who lack a prior sample to act as a standard or for routine screening of the general public. This type of method can also allow standardized tests to be performed among several clinics, institutions, or countries etc. A standard used in this manner can provide information about an individual's risk of developing a macrophage-mediated disease in a manner similar to the cardiovascular risk that is assessed using routine monitoring of cholesterol and C-reactive protein in a blood sample.

The reference series may also include sample values of sCD163 in individuals at various stages of the macrophage-mediated disease. Thus, reference values for individuals with early onset dementia or heart disease may provide a stage-specific diagnosis or timeline for diagnosis if such is desired.

### ART Therapy

In some embodiments, the methods described herein are useful for analyzing HIV+ individuals who are being treated with anti-retroviral therapy (ART). Similarly, the assays and methods in some embodiments include administering therapy to the individual if they have been diagnosed as being at risk of developing a macrophage-mediated diseases.

Treatments may include any number of anti-retroviral therapies. A number of reverse transcriptase inhibitors are commercially available. Examples include, but are not limited to, nucleoside analogs, which are a class of compounds that are known to inhibit HIV, and non-nucleoside drugs. Nucleoside analogs are exemplified by didanosine (2′,3′-dideoxyinosine or [ddl], available as VIDEX® from Bristol Myers-Squibb, Wallingford, Conn.); zidovudine (3′-azido-2′,3′-dideoxythymidine or azidothymidine [AZT], available from Glaxo-Wellcome Co., Research Triangle Park, N.C.); zalcitabine (2′,3′-dideoxycytidine [ddC], available as HIVID® from Hoffman-La Roche, Basel, Switzerland); lamivudine 2′-deoxy-3′-thiacytidine [3TC] (EPIVIR®, available from Glaxo-Wellcome Co.); stavudine (2′,3′-didehydro-2′,3′-dideoxythimidine [D4T] available as ZERIT®) from Bristol Myers-Squibb); and the combination drug zidovudine plus lamivudine (COMBIVIR®, available from Glaxo Wellcome). These particular drugs belong to the class of compounds known as 2′,3′-dideoxynucleoside analogs, which, with some exceptions such as 2′,3′-dideoxyuridine [DDU], are known to inhibit HIV replication, but have not been reported to clear any individual of the virus. Other nucleoside reverse transcriptase inhibitors include abacavir (1592U89, ZIAGEN™, available from Glaxo-Wellcome Co.)—Non-nucleoside reverse transcriptase inhibitors include nevirapine (VIRAMUNE™, available from Boehringer Ingelheim Pharmaceuticals, Inc.); delaviridine (RESCRIPTOR®, available from Pharmacia & Upjohn, Kalamazoo, Mich.); and efavirenz (available as SUSTIVA®, from DuPont Merck).

Examples of protease inhibitors that can be used to treat individuals with HIV infection include, but are not limited to, Indinavir sulfate (available as CRIXIVAN™ capsules from Merck & Co., Inc., West Point, Pa.), saquinavir (INVIRASE® and FORTOVASE®, available from Hoffmnan-La Roche), ritonavir (NORVIR®, available from Abbott Laboratories, Abbott Park, 111.); ABT-378 (new name: lopinavir, available from Abbott Laboratories); Amprenavir (AGENERASE™, available from Glaxo Wellcome, Inc.); and Nelfinavir (VIRACEPT®), and GW141 (available from Glaxo Wellcome/Vertex). Such examples of reverse transcriptase and protease inhibitors are not intended to be limiting. It is recognized that any known inhibitor, as well as those under development, may be used in combination with the methods described herein.

Suitable human dosages for these compounds vary widely. However, such dosages can readily be determined by those of skill in the art. Therapeutically effective amounts of these drugs are administered. “Therapeutically effective amount” is intended to refer to an amount of the antiretroviral agent that is sufficient to decrease the effects of HIV infection, or an amount that is sufficient to favorably influence the pharmacokinetic profile of one or more of the other antiretroviral agents used. In one embodiment, a therapeutically effective amount is an amount that reduces sCD163 levels by at least 10% compared to the levels prior to treatment. Decrease in dosage frequency can be advantageous for antiretroviral agents having undesirable side effects when administered in the absence of the antiretroviral agent that increases their bioavailability.

In some embodiments, the dosages can be optimized by using the methods and assays of the invention to determine if the dosage reduces the amount of sCD163. So, for example, a first biological sample can be taken prior to administration of a therapy or a test therapy and sCD163 can be measured in that sample to provide a baseline amount, then the therapy or test therapy can be administered at a first amount or dose, a second biological sample can be taken from the individual after a time period on the therapy, and sCD163 is again measured in the second biological sample. If the sCD163 is decreased in the second biological sample, the therapy may be considered to work. If the sCD163 is not decreased or is decreased only marginally, one can increase the dose of the therapeutic or alter it otherwise. The effect of such dosage change or therapy alteration can then be monitored by another measurement of the sCD163 level in the HIV-positive individual.

In one embodiment, an antiretroviral agent, when administered in a therapeutically effective amount to an HIV-infected subject, decreases the effects of HIV infection by, for example, inhibiting replication of HIV, thereby decreasing viral load in the subject undergoing antiretroviral therapy. In another embodiment, an antiretroviral agent, when administered in a therapeutically effective amount to an HIV-infected subject, favorably influences the pharmacokinetics of one or more of the other antiretroviral agents used.

### Systems

Embodiments of the invention also provide for systems (and computer readable media for causing computer systems) to perform a method for assessing a subject's risk of developing a macrophage-mediated disorder in a subject infected with HIV, or monitoring efficacy of a treatment for a macrophage-mediated disorder administered to a subject infected with HIV.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media #30 can be any available tangible media that can be accessed by a computer. Computer readable storage media includes volatile and nonvolatile, removable and non-removable tangible media implemented in any method or technology for storage of information such as computer readable instructions, data structures, program modules or other data.

Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable storage media may define instructions, for example, as part of one or more programs, that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable storage media on which such instructions are embodied may reside on one or more of the components of either of a system, or a computer readable storage medium described herein, may be distributed across one or more of such components.

The computer-readable storage media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

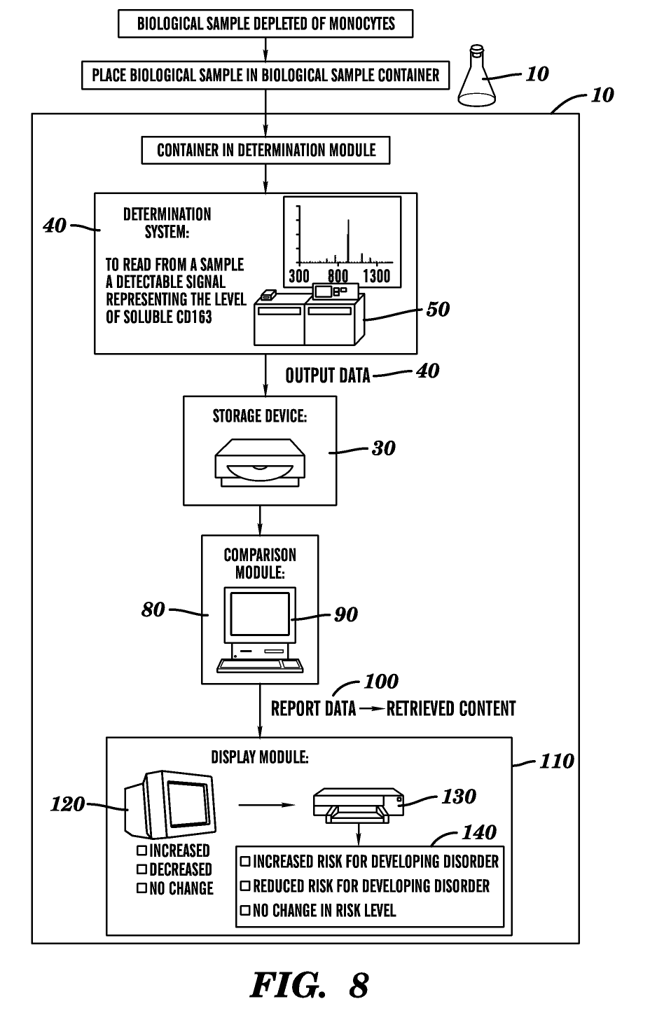

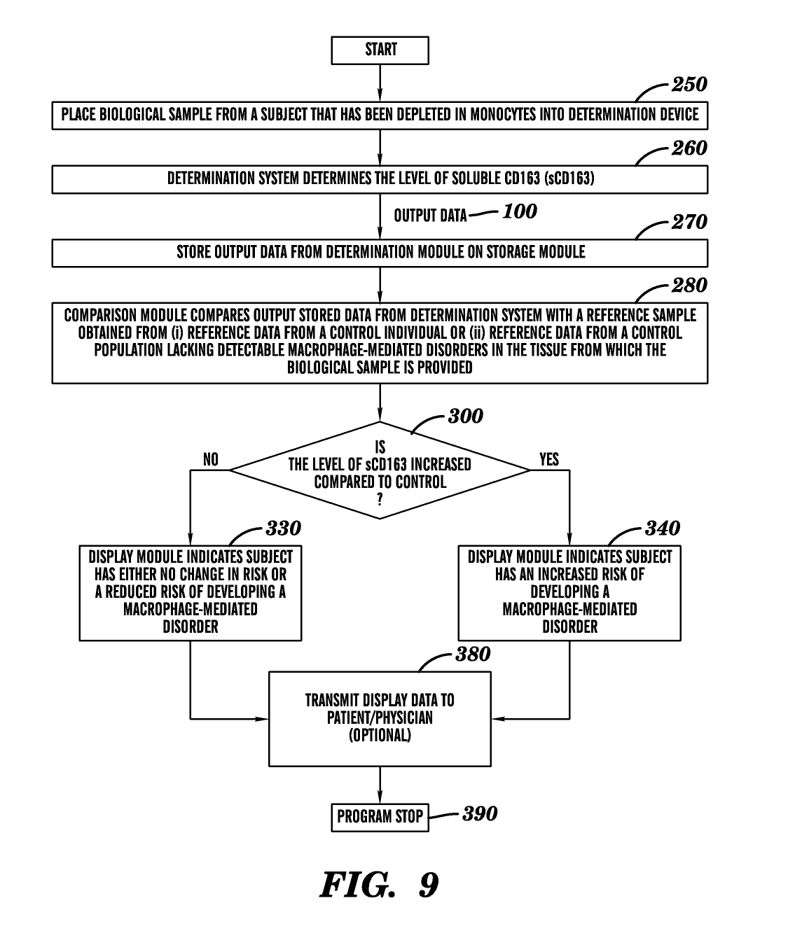

The functional modules of certain embodiments of the invention include at minimum a determination system #40 (FIG. 8), a storage device #30 (FIG. 8), a comparison module #80 (FIG. 8), and a display module #110 (FIG. 8). The functional modules can be executed on one, or multiple, computers, or by using one, or multiple, computer networks. The determination system has computer executable instructions to provide e.g., sequence information in computer readable form.

The determination system #40 (FIG. 8), can comprise any system for detecting a signal representing the level of sCD163. Such systems can include DNA microarrays, RNA expression arrays, PCR etc.

The information determined in the determination system can be read by the storage device #30. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon nucleic acid sequence information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising information relating to sCD163 level.

In one embodiment the reference data stored in the storage device to be read by the comparison module is e.g., sequence data obtained from a subject at an earlier time point or a population of subjects infected with HIV.

The “comparison module” #80 (FIG. 8) can use a variety of available software programs and formats for the comparison that operate in comparing sequence information data determined in the determination system to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to the level of sCD163 in an individual.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that may be stored and output as requested by a user using a display module #110 (FIG. 8).

The content based on the comparison result, may be an increase in the level of sCD163 indicating an increased risk of a macrophage-mediated disorder. Alternatively, the content based on the comparison result may be a decrease in the level of sCD163 indicating a reduction in risk or efficacious treatment of the macrophage-mediated disease.

In one embodiment of the invention, the content based on the comparison result is displayed on a computer monitor #120. In one embodiment of the invention, the content based on the comparison result is displayed through printable media #130, #140 (FIG. 8). The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The methods described herein therefore provide for systems (and computer readable media for causing computer systems) to perform methods for assessing risk or monitoring treatment of a macrophage-mediated disorder in an HIV-positive individual.

Systems and computer readable media described herein are merely illustrative embodiments of the invention for performing methods of diagnosis in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

In one aspect, provided herein is an assay for determining risk for onset of a macrophage-mediated disease in an individual infected with HIV, comprising the steps of: (a) contacting a first biological sample comprising plasma, blood, or cerebral spinal fluid from the individual with an antibody against at least one soluble extracellular domain of CD163 and measuring the amount of soluble CD163 in the first biological sample; (b) contacting a second biological sample comprising plasma, blood, or cerebral spinal fluid from the individual obtained at a subsequent time period with an antibody against the at least one extracellular domain of CD163 and measuring the amount of soluble CD163 in the second biological sample, and (c) comparing the difference in the amount of soluble CD163 between the first and second biological sample, wherein the individual is at risk for onset of a macrophage-mediated disease if the amount of soluble CD163 is increased by at least 10% in the second biological sample.

In another aspect, described herein is an assay for determining or monitoring the effectiveness of a treatment of a macrophage-mediated disease in an individual infected with HIV, comprising the steps of: (a) contacting a first biological sample comprising plasma, blood, or cerebral spinal fluid from an individual infected with HIV and further affected with a macrophage-mediated disease with an antibody against at least one extracellular domain of CD163 and measuring the amount of soluble CD163 in the first biological sample, (b) administering a treatment to the individual, (c) contacting a second biological sample comprising plasma, blood, or cerebral spinal fluid from the individual obtained after administration of the treatment with an antibody against at least one soluble extracellular domain of CD163 and measuring the amount of soluble CD163 in the second biological sample, and (d) comparing the difference in the amount of soluble CD163 between the first and second biological sample, wherein the treatment is effective if the amount of soluble CD163 is decreased by at least 10% in the second biological sample. If the treatment is not effective, i.e. the sCD163 level is not lowered, one can adjust the dosage of the medicine. The assay can be repeated after adjustment of a dosage and if not effective, the treatment can be changed to an alternative treatment.

Also provided herein, in another aspect, is an assay for screening for an agent to treat a macrophage-mediated disease in a test model infected with HIV or simian immune deficiency (SIV), comprising the steps of: (a) contacting a first biological sample comprising plasma, blood, or cerebral spinal fluid from a test model infected with HIV or SIV and further affected with a macrophage-mediated disease with an antibody against at least one extracellular domain of CD163 and measuring the amount of soluble CD163 in the first biological sample, (b) administering a treatment to the test model, (c) contacting a second biological sample comprising plasma, blood, or cerebral spinal fluid from the test model obtained after administration of the treatment with an antibody against the at least one soluble extracellular domain of CD163 and measuring the amount of soluble CD163 in the second biological sample, and (d) comparing the difference in the amount of soluble CD163 between the first and second biological sample, wherein the treatment is effective if the amount of soluble CD163 is decreased by at least 10% in the second biological sample.

In one embodiment of the aspects described herein, the biological sample is plasma.

In another embodiment of the aspects described herein, the biological sample is blood.

In another embodiment of the aspects described herein, the biological sample is cerebral spinal fluid (CSF).

In some embodiments, instead of an antibody, one can measure the amount of sCD163 mRNA to determine the amount of sCD163 in the sample.

Another aspect provided herein relates to an assay for determining risk of HIV infection in an individual that has not undergone seroconversion, comprising the steps of: (a) contacting a first biological sample comprising plasma or blood from the individual with an antibody against at least one extracellular domain of CD163 and measuring the amount of soluble CD163 in the first biological sample; (b) contacting a second biological sample comprising plasma or blood with an antibody against the at least one soluble extracellular domain of CD163 and measuring the amount of soluble CD163 in the second biological sample, and (c) determining the difference in the amount of soluble CD163 between the first and second biological sample, wherein the individual is at risk for HIV infection if the amount of soluble CD163 is increased by at least 10% in the second biological sample.

An additional aspect provided for herein relates to a method for determining risk of HIV infection in an individual that has not undergone seroconversion, comprising the steps of: (a) transforming a first and second biological sample comprising blood from an individual infected with HIV to deplete the first and second biological sample of monocytes, and (b) determining risk for HIV infection using an assay described herein.

In another aspect, provided herein is an assay for determining risk for onset of a macrophage-mediated disease in an individual infected with HIV, comprising the steps of: (a) contacting a biological sample comprising plasma, blood, or cerebral spinal fluid from the individual with an antibody against at least one soluble extracellular domain of CD163 and measuring the amount of soluble CD163 in the biological sample; and (b) comparing the difference in the amount of soluble CD163 between the biological sample obtained from the individual to a standard, such as a population standard, wherein the individual is at risk for onset of a macrophage-mediated disease if the amount of soluble CD163 is increased by at least 10% in the biological sample compared to the standard.

In some embodiments of the aspects described herein, the population standard comprises an average sCD163 level in a population of HIV+ individuals or individuals not infected with HIV. In some embodiments, the standard is an average sCD163 level in HIV infected individuals with no symptoms of other macrophage-associated disease.

In another embodiment of the aspects described herein, the macrophage-mediated disease is selected from the group consisting of: AIDS-related dementia, peripheral neuropathy, and HIV-associated heart disease (e.g., coronary atherosclerosis).

In one embodiment, the macrophage-mediated disease is coronary atherosclerosis. In some aspects of the invention, when the disease is coronary atherosclerosis, the individual is an HIV+ male individual.

In another embodiment, sCD163 levels are correlated with coronary artery plaques (non-calcified).

In another embodiment of the aspects described herein, the blood sample is a monocyte-depleted sample.

In another embodiment of the aspects described herein, the individual has detectable HIV viral levels.

The time interval between the first and the second or subsequent samples can be any interval. In another embodiment of the aspects described herein, the interval between the samples is 1 week.

In another embodiment of the aspects described herein, the interval between the samples is, for example, the second biological sample is obtained at least 1 month after the first biological sample.

In other embodiments of the aspects described herein, the interval between the samples is 3, 6, 9, or 12 months, or 2, 3, 4, 5, 6, 7, 8, 9, 10 or more years. For example, the second biological sample can be obtained at least 3 months, at least 6 months, at least 9 months, at least 1 year, at least 2 years, at least 5 years, at least 10 years or more after the first biological sample.

In another embodiment of the aspects described herein, the test model is a mammalian model.

In another embodiment of the aspects described herein, the mammalian model is a non-human primate model. In some embodiments, the mammalian model is human.

In another embodiment of the aspects described herein, the measuring step is performed using ELISA techniques.

In some embodiments of the aspects described herein, the measuring step if performed using mRNA quantification techniques, such as real time RT-PCR and other well known methods, wherein one designs the pripers to be specific to sCD163, so as to not amplify macrophage-associated CD163.

In another embodiment of the aspects described herein, the antibody does not measure macrophage-associated CD163.

In another embodiment of the aspects described herein, the first and/or second and/or subsequent biological sample is stored before the assays or methods.

In another embodiment of the aspects described herein, the first and/or second and/or a subsequent biological sample is frozen or lyophilized.

Another aspect provided herein relates to a computer readable storage medium having computer readable instructions recorded thereon to define software modules for implementing on a computer a method for assessing soluble CD163 levels in a first and/or second and/or a subsequent monocyte-depleted sample, said computer readable storage medium comprising: (a) instructions for storing and accessing data representing a level of soluble CD163 determined for a first and/or second monocyte-depleted sample obtained from an individual infected with HIV; (b) instructions for comparing said level of said soluble CD163 in the second and or subsequent monocyte-depleted sample to the level of soluble CD163 in the first monocyte-depleted sample or to a reference sample, whereby a change in the level of soluble CD163 is determined, (c) instructions for displaying retrieved content to a user, wherein the retrieved content comprises an increase, decrease or a value of the level of soluble CD163 in the second biological sample compared to the level of soluble CD163 in the first biological sample or reference sample.

Another aspect described herein relates to a computer system for obtaining data from at least one monocyte-depleted sample comprising plasma obtained from at least one individual, the system comprising: (a) a specimen container to hold the at least one sample; (b) a determination module configured to determine read-out information, wherein said read-out information comprises information representing an amount of soluble CD163 in the at least one sample, and (c) a storage device configured to store data output from said determination module, (d) comparison module adapted to compare the data obtained from the determination module with reference data on said storage device, whereby a change in the level of soluble CD163 is determined, and (e) a display module for displaying retrieved content to the user, wherein the retrieved content comprises an increase, decrease or value for soluble CD163 in the at least one monocyte-depleted sample compared to a reference sample.

In another embodiment of the aspects described herein, the reference sample is a different monocyte-depleted sample from the same individual, from a different individual, or from a population of individuals infected with HIV.

In another embodiment of the aspects described herein, the reference sample is a numerical value.

Another aspect provided herein relates to a method for determining risk of onset of a macrophage-mediated disease in an individual infected with HIV, comprising the steps of: (a) contacting a sample obtained from an individual infected with HIV at a first time point with an antibody against soluble CD163 and measuring the amount of soluble CD163 at the first time point; (b) contacting a sample obtained from the individual infected with HIV at at least a second time point with an antibody against soluble CD163 and measuring the amount of soluble CD163 at the second time point, and (c) determining the difference in the amount of soluble CD163 between the first and the at least second time point, wherein the individual is at the risk of onset of a macrophage-mediated disease if the amount of soluble CD163 is increased by at least 10% at the at least second time point.

Also described herein is a method for monitoring the effectiveness of a treatment of a macrophage-mediated disease in an individual infected with HIV, comprising the steps of: (a) contacting a first sample taken at a first time point obtained from an individual infected with HIV and further affected with a macrophage-mediated disease with an antibody against soluble CD163, (b) administering a treatment to the patient, (c) contacting a second sample taken at, at least a second time point obtained from the individual after administration of the treatment with an antibody against soluble CD163, and (d) determining the difference in the amount of soluble CD163 between the first and the at least second time point; wherein the treatment is effective if the amount of soluble CD163 is decreased by at least 10% at the at least second time point.

Also provided herein is a method for screening for an agent to treat a macrophage-mediated disease in a test model infected with HIV or SIV, comprising the steps of: (a) contacting a first sample taken at a first time point obtained from a test model infected with HIV or SIV and further affected with a macrophage-mediated disease with an antibody against soluble CD163, (b) administering a treatment to the test model, (c) contacting a second sample taken at at least a second time point obtained from the test model after administration of the treatment with an antibody against soluble CD163, and (d) determining the difference in the amount of soluble CD163 between the first and second time point; wherein the treatment is effective if the amount of soluble CD163 is decreased by at least 10% at the second time point.

In some embodiments, the invention provides a computer readable storage medium having computer readable instructions recorded thereon to define software modules for implementing on a computer a method for assessing soluble CD163 levels in a first and/or second monocyte-depleted sample, said computer readable storage medium comprising:

(a) instructions for storing and accessing data representing a level of soluble CD163 determined for a first and/or second monocyte-depleted sample obtained from an individual infected with HIV;

(b) instructions for comparing said level of said soluble CD163 in the second monocyte-depleted sample to the level of soluble CD163 in the first monocyte-depleted sample or to a reference sample, whereby a change in the level of soluble CD163 is determined,

(c) instructions for displaying retrieved content to a user, wherein the retrieved content comprises an increase, decrease or a value of the level of soluble CD163 in the second biological sample compared to the level of soluble CD163 in the first biological sample or reference sample.

In another embodiment, the invention provides a computer system for obtaining data from at least one monocyte-depleted sample comprising plasma obtained from at least one individual, the system comprising: (a) a specimen container to hold the at least one sample; (b) a determination module configured to determine read-out information, wherein said read-out information comprises information representing an amount of soluble CD163 in the at least one sample; (c) a storage device configured to store data output from said determination module, (d) comparison module adapted to compare the data obtained from the determination module with reference data on said storage device, whereby a change in the level of soluble CD163 is determined; and (e) a display module for displaying retrieved content to the user, wherein the retrieved content comprises an increase, decrease or value for soluble CD163 in the at least one monocyte-depleted sample compared to a reference sample.

In some aspects of this embodiment and all the embodiments of the invention, the reference sample is a different monocyte-depleted sample from the same individual, from a different individual, or from a population of individuals infected with HIV.

In some aspects of this embodiment and all the embodiments of the invention, the reference sample is a numeric value.

In some aspects of this embodiment and all the embodiments of the invention, the HIV-associated heart disease is coronary atherosclerosis, HIV-associated dementia, or HIV-associated cardiomyopathy.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified in the specification and examples are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All the references cited herein and throughout the specification are incorporated by reference in their entirety.

### EXAMPLES

### Example 1

CD163, a monocyte/macrophage-specific scavenger receptor, is shed during activation as soluble CD163 (sCD163). We have previously demonstrated that monocyte expansion from bone marrow with SIV infection correlated with plasma sCD163, the rate of AIDS progression and severity of macrophage-mediated pathogenesis. Here we examined sCD163 in HIV infection. sCD163 was elevated in plasma of chronically (>1 year) infected individuals compared to HIV-seronegatives. With effective antiretroviral therapy (ART), sCD163 declined in parallel to HIV-RNA, but did not return to HIV-seronegative levels, suggesting the presence of residual monocyte/macrophage activation even with plasma virus below the limit of detection. In early HIV-infected individuals (<1 year), effective ART resulted in decreased sCD163 comparable to HIV-seronegative levels. sCD163 in plasma positively correlated with the percentage of CD14+ CD16+ monocytes, activated CD8+ HLA-DR+CD38+ T lymphocytes and inversely with CD163 expression on CD14+ CD16+ monocytes. With ART interruption in early HIV-infected subjects, sCD163 and plasma virus spiked but rapidly returned to baseline with ART re-initiation. This study points to the utility of monocyte/macrophage-derived sCD163 as a marker of HIV activity that links viral replication with monocyte/macrophage activation. These observations underscore the significance of monocyte/macrophage immune responses with HIV pathogenesis.

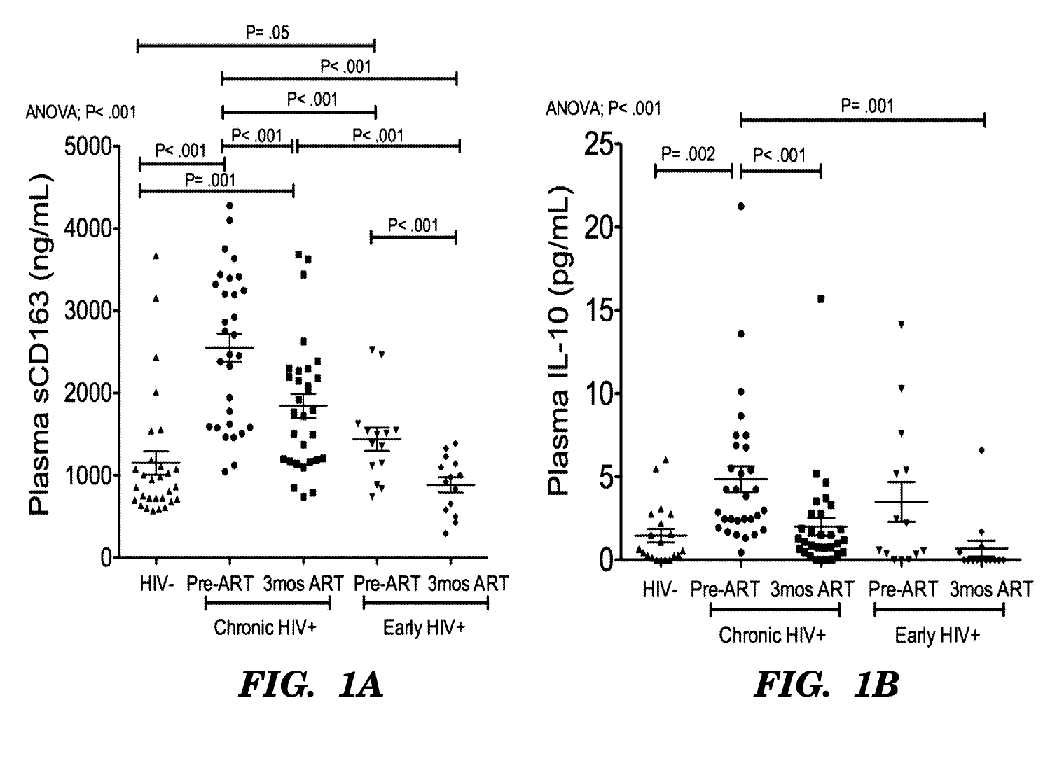

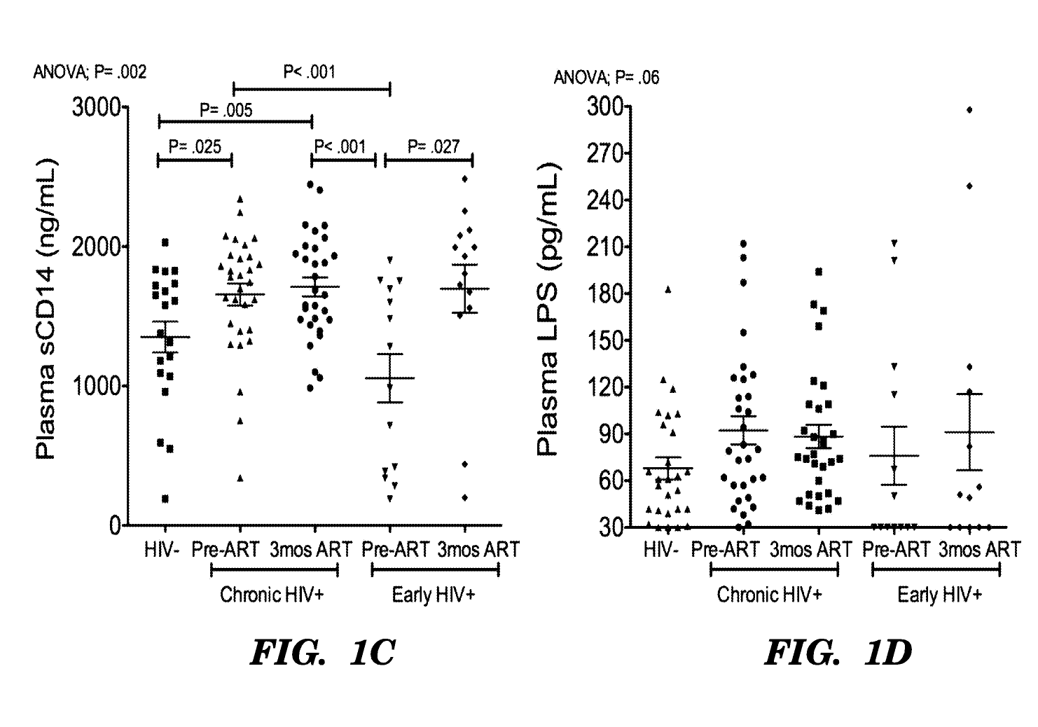

Plasma was examined from 30 chronically HIV-infected individuals (at least 1 year since self-reported seropositive HIV test) at two points, before (Chronic HIV+, Pre-ART) and after 3 months of successful antiretroviral therapy (ART) (Chronic HIV+, 3mos ART). sCD163 levels in HIV-infected subjects were compared to age-matched HIV-seronegative individuals (FIG. 1A). Parallel to our studies in chronically HIV-infected individuals, a cohort of 14 HIV-infected subjects that were identified during acute (before seroconversion) or early infection (the first year of infection defined by the appearance of HIV-specific antibodies as identified by an indeterminate or positive Western blot [32-34]) were examined for sCD163 in plasma. Samples were examined during early HIV infection (≦1 year) when patients were naïve to ART and after 3 months of ART (FIG. 1A). Plasma sCD163 was significantly different among the groups (ANOVA, P<0.0001). sCD163 in plasma was elevated in chronically HIV-infected individuals before (Pre-ART) and after 3 months of ART (3mos ART) compared to HIV-seronegatives (HIV-) (FIG. 1A, P=4×10⁻⁸ and P=0.001, respectively). We found a significant decrease in sCD163 in plasma after 3 months of ART in the chronically HIV-infected group (FIG. 1A, paired t-test P=9×10⁻⁸), although sCD163 did not return to HIV-seronegative levels (FIG. 1A), suggesting residual monocyte activation persists even with undetectable plasma viral load. Elevated sCD163 in plasma is consistent with monocyte activation and stimulation of innate immunity with HIV infection. sCD163 levels in early HIV-infected subjects were higher than those in HIV-seronegative individuals (FIG. 1A, P=0.05) but lower than in chronic HIV-infected individuals (P=0.0002). After ART, sCD163 in early HIV infection returned to levels seen in HIV-seronegative individuals (FIG. 1A, P=0.32). Examining samples from the same early HIV-infected individuals Pre-ART and 3mos ART, we found that sCD163 declined with ART that was initiated within the first year after seroconversion (FIG. 1A, paired t test, P=0.0007). These data suggest that ART in early HIV-infected patients decreases plasma virus and may lower monocyte/macrophage activation as shown by decrease in CD163 shedding.

Since IL-10 is an inflammatory mediator expressed by monocytes as well as many other cell types [35], IL-10 plasma levels were assessed. IL-10 levels were significantly different among groups (ANOVA, P<0.0001). IL-10 was elevated in chronically HIV-infected subjects (Pre-ART) compared with HIV-seronegative individuals (FIG. 1B, P=0.002). Following ART, IL-10 plasma levels dropped significantly (FIG. 1B, paired t-test P=7×10⁻⁶) to levels similar to HIV-seronegatives (FIG. 1B, P=0.47). Mean plasma IL-10 levels were similar in early HIV infection and in HIV-seronegatives (FIG. 1B, P=0.07). There was no significant change in IL-10 after 3 months of ART (FIG. 1B, paired t-test, P=0.06).

Increased sCD14 and LPS in plasma are associated with immune activation in HIV-infected individuals and possibly HIV pathogenesis and therefore were assessed (FIG. 1C-D). Plasma sCD14 levels were significantly different among groups (ANOVA, P 0.002). sCD14 in chronically HIV-infected individuals before and after ART was elevated compared with HIV-seronegative individuals (FIG. 1C, P=0.025 and P=0.005, respectively). Interestingly, sCD14 levels in early HIV-infected subjects pre-ART were equivalent to HIV seronegatives, but were significantly elevated after 3mos ART (FIG. 1C, P=0.027). Plasma LPS levels were not significantly different among groups (ANOVA, P 0.06) (FIG. 1D). However, there was a trend for higher LPS in the chronic patients pre-ART compared to HIV-seronegatives. Additional factors produced by macrophages, including plasma IL-6 and osteopontin, were not significantly altered with HIV infection or ART.

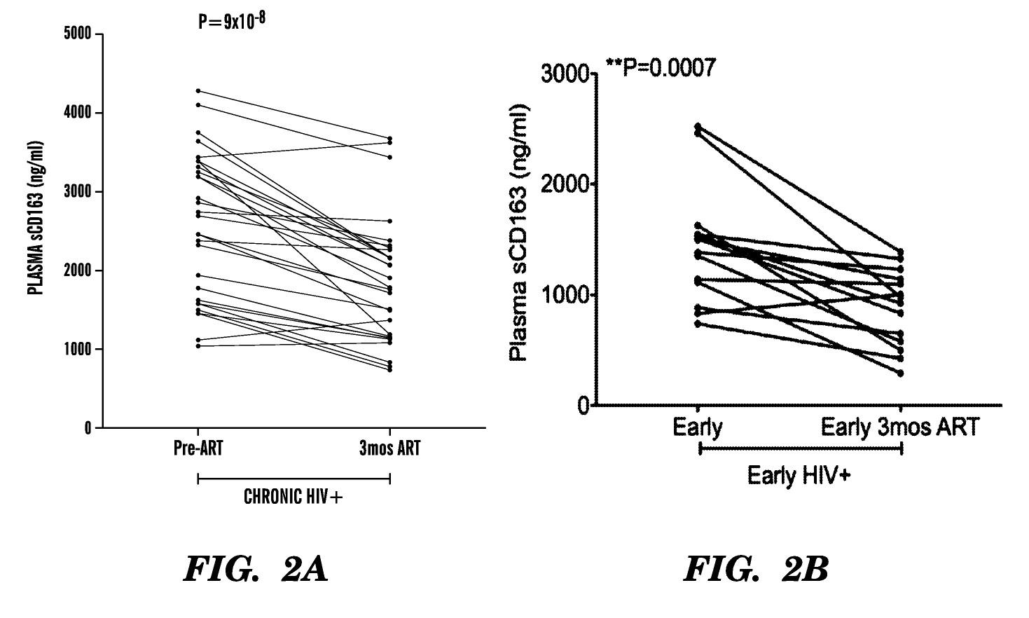

Additional factors produced by macrophages, plasma IL-6 and osteopontin (OPN), were not changed with HIV infection in this cohort of patients. Next, the effect of ART on these markers was assessed in individual HIV-infected subjects prior to and after 3 months of ART, when plasma viral load was undetectable. A significant decrease in sCD163 and IL-10 in plasma in chronically HIV-infected individuals after 3 months of ART was observed (FIG. 2A, paired t-test P=9×10⁻⁸). ART did not reduce plasma levels of sCD14, LPS, IL-6 or osteopontin (OPN).

The percent change of sCD163 positively correlated to the percent change in plasma viral load (P=0.007, r=0.48) and negatively correlated to percent change of absolute numbers of CD4+ T lymphocyte (P=0.02, r=−0.42). As patients improved with ART, sCD163 levels dropped. In chronic HIV-infected patients, monocyte numbers correlated with plasma sCD163 prior to ART (P=0.05, r=0.36) but were not maintained with therapy (P=0.20, r=0.24).

To further study plasma sCD163 as a functional marker of progressive HIV infection, virological and hematological parameters were examined that have been previously used to link disease progression or assess efficacy of treatment, including plasma viral load, absolute numbers of CD4+ T lymphocytes, monocytes and platelets. In addition, hemoglobin levels and hematocrit, both linked to macrophage scavenging functions and possibly to plasma sCD163, were examined. The percent change of sCD163 positively correlated to the percent change in plasma viral load (P=0.007, r=0.48). Following ART, there was a decrease in both sCD163 and plasma viral load.

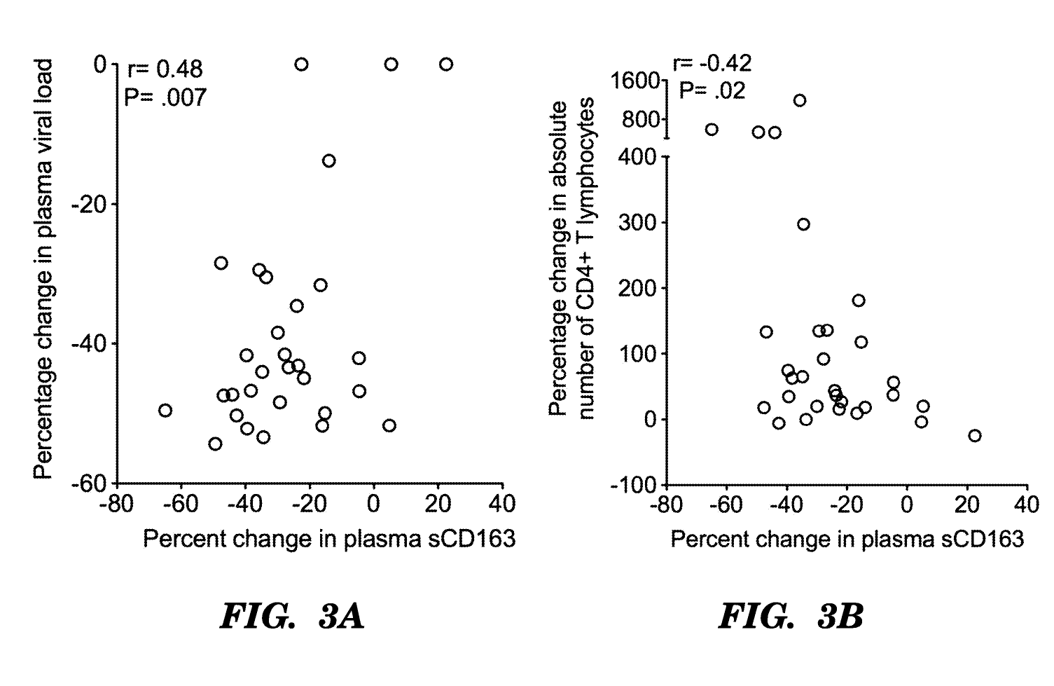

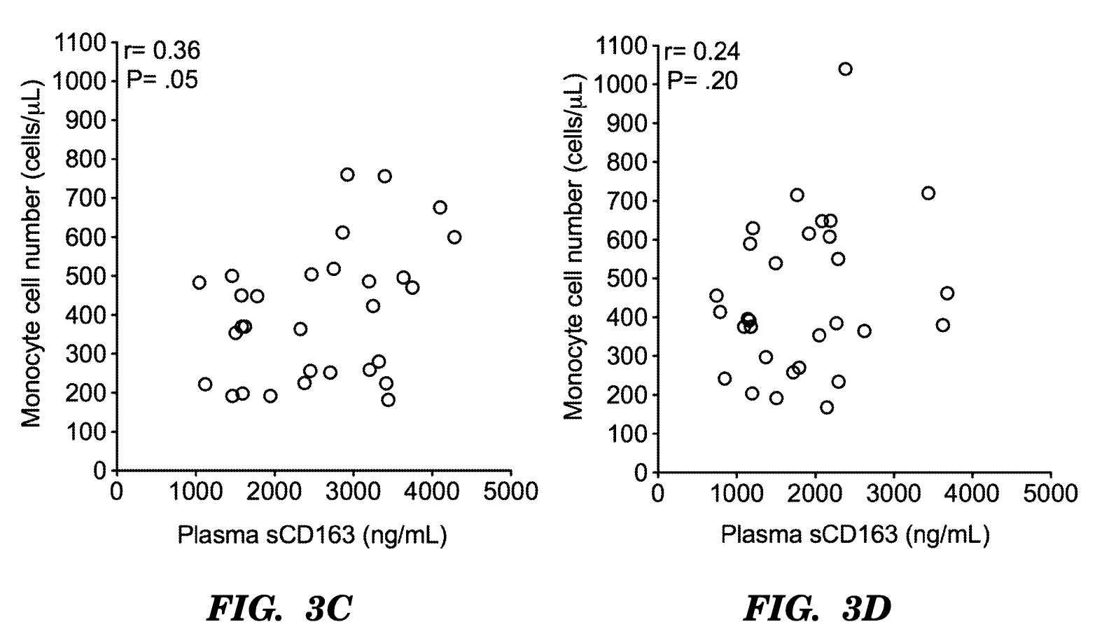

Next, we examined correlations of sCD163 with plasma virus, lymphocytes and monocytes. The percent change of sCD163 positively correlated to the percent change in plasma viral load (FIG. 3A, P=0.007, r=0.48) and negatively correlated to percent change of absolute numbers of CD4+ T lymphocyte (FIG. 3B, P=0.02, r=−0.42). As patients improved with ART, sCD163 levels dropped. In chronic HIV-infected patients, monocyte numbers correlated with plasma sCD163 prior to ART (FIG. 3C, P=0.05, r=0.36) but were not maintained with therapy (FIG. 3D, P=0.20, r=0.24).

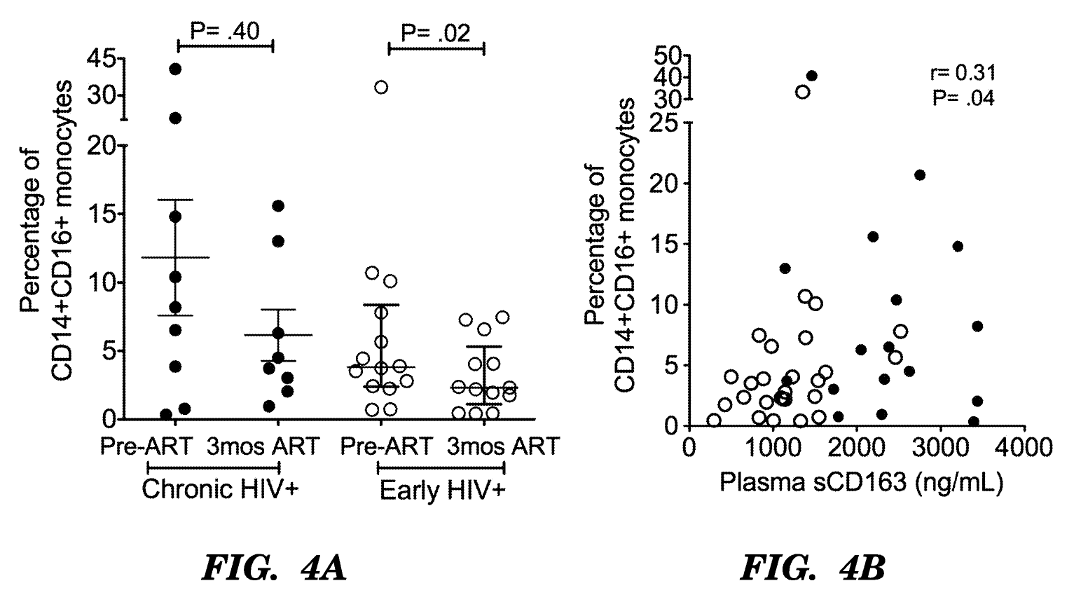

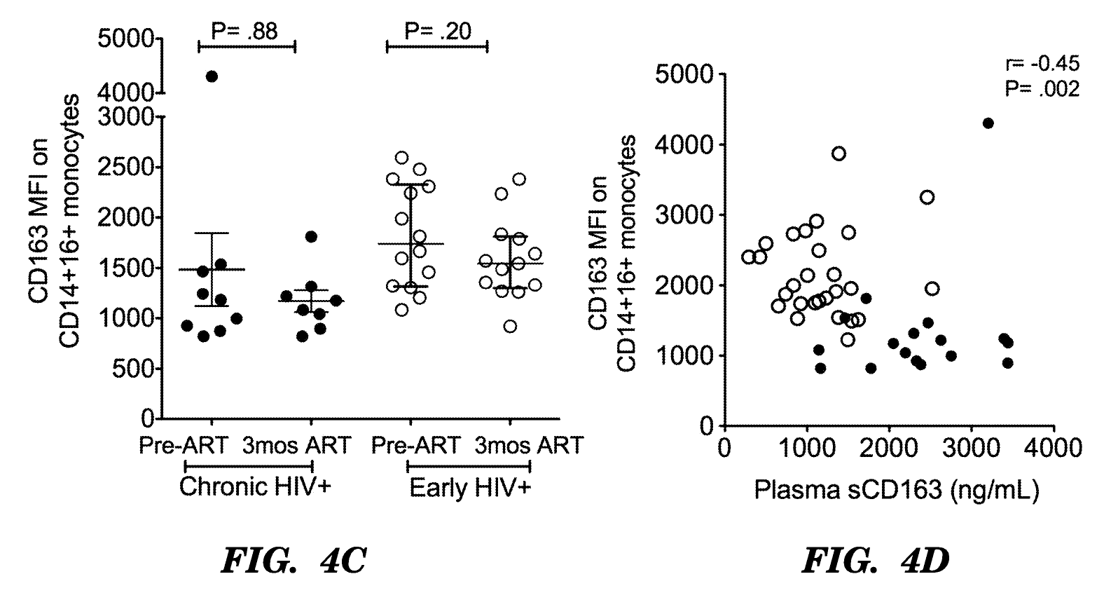

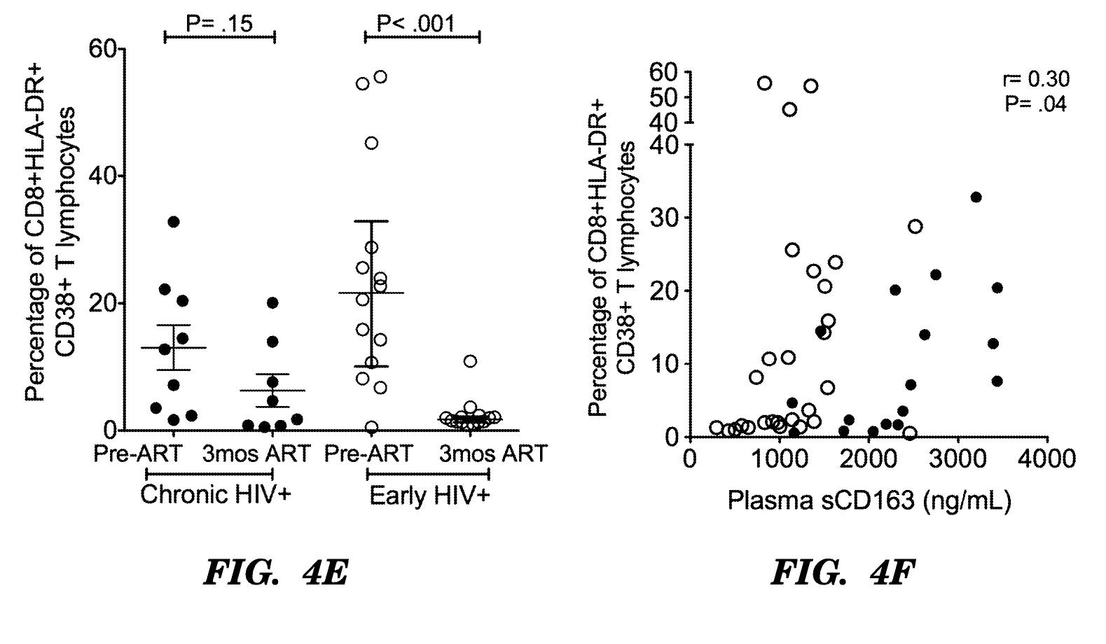

We determined if there was a correlation between sCD163 with increased percentage of activated CD14+ CD16+ monocytes or CD163 expression on monocytes using PBMC from a subset of chronically HIV-infected patients (8 Pre-ART and 9 3mos ART, with 2 matched pairs) and all early HIV-infected patients. PBMC samples and plasma used for sCD163 assays were taken simultaneously (FIG. 4). As others have shown, we found a trend for a decreased percentage of CD14+ CD16+ monocytes in the chronically HIV-infected patients after ART, that did not reach significance (FIG. 4A, closed circles) and a significant decreased percentage of CD14+ CD16+ monocytes in the early HIV-infected patients after ART (FIG. 4A, open circles, paired t-test P=0.02). The percentage of CD14+ CD16+ monocytes in all HIV-infected patients correlated with plasma sCD163 levels (FIG. 4B, overall r=0.31, P=0.04). When early and chronic patients were examined separately a significant correlation was found in the early HIV-infected population but not in the chronic population (FIG. 4B, open circles, r=0.40, P=0.03). There were no significant changes in the median fluorescence intensity (MFI) of CD163 on CD14+ CD16+ monocytes after ART (FIG. 4C). Interestingly, there was an inverse correlation between CD163 expression on CD14+ CD16+ monocytes and sCD163 in plasma (FIG. 4D, overall r=−0.45, P=0.002), although this negative correlation was not apparent when the early and chronic patients were examined separately. In early HIV-infected individuals, there are low levels of sCD163 in plasma, but high CD163 levels on CD14+ CD16+ monocytes. In the chronic HIV-infected individuals, there are high levels of sCD163 in plasma, but low CD163 levels on CD14+ CD16+ monocytes. These data support the notion that sCD163 is shed from CD14+ CD16+ monocytes.

Next, we measured standard markers of immune activation (CD8+ HLA-DR+CD38+ T lymphocytes) to assess if shedding of sCD163 is associated with immune activation (FIG. 4E, 4F). There was a significant decrease of CD8+ HLA-DR+CD38+ activated T lymphocytes in the early HIV-infected group after ART (FIG. 4E, open circles, paired t-test P=0.0005). Similarly, there was a decrease, although not significant, in the chronically HIV-infected individuals after ART (FIG. 4E, closed circles, P=0.15). Overall, sCD163 significantly correlated with the percentage of CD8+ HLA-DR+CD38+ T lymphocytes (FIG. 3F, overall r=0.30, P=0.04). Within groups, sCD163 significantly correlated with the percentage of CD8+ HLA-DR+CD38+ T lymphocytes in both early (FIG. 4F, open circles, r=0.45, P=0.02) and chronic (FIG. 4F, closed circles, r=0.61, P=0.01) subjects. Thus, shedding of sCD163 is strongly associated with immune activation.

Without wishing to be bound by theory, it is possible that levels of sCD163 may correlate with the expansion of a specific monocyte subset, such as CD14+ CD16+ monocytes, numbers of which may decrease with successful ART. PBMCs were not available to examine monocyte subsets retrospectively. In SIV infection, a significant correlation was not found between increased plasma sCD163 and an increased percentage and absolute number of CD14+ CD16+ monocytes (FIG. 8A, P=0.002, r=0.53 and 1B, P=0.02, r=0.43).

Parallel to the studies in chronically HIV-infected individuals, plasma sCD163 and IL-10 levels were investigated in a cohort of 14 HIV-infected subjects that were identified during acute (before seroconversion) or early infection (the first year of infection defined by the appearance of HIV-specific antibodies as identified by an indeterminate or positive Western blot (Kassutto, S., and Rosenberg, E. S. 2004. Glen Infect Dis 38: 1447-1453; Alter, G., et al., 2007. J Infect Dis 195:1452-1460; Lentz, M. R., et al., 2009. Neurology 72:1465-1472). Samples from these subjects were examined during early HIV infection (≦1 year) when naïve to ART and after 3 months of ART. sCD163 levels in early HIV-infected subjects were significantly higher than those in HIV-seronegative individuals (P=0.05) but lower than in chronic HIV-infected individuals (P=0.0002). After ART, sCD163 in early HIV infection returned to levels seen in HIV-seronegative individuals (P=0.32). Examining matched samples of early HIV-infected subjects, it was found that sCD163 declined with ART that was initiated within the first year after seroconversion (FIG. 2B, paired t test, P=0.0007). These data indicate that ART in early HIV-infected patients may lower monocyte/macrophage activation and CD 163 shedding as well as decrease plasma viral load and lymphocytes.

Mean plasma IL-10 levels were similar in early HIV infection and in HIV-seronegatives (P=0.07). Additionally, in the early HIV-infected subjects examined here, there was not a change in IL-10 after 3 months of ART (paired t-test, P=0.06). Again, sCD163 was a more reliable marker of macrophage activation and successful ART therapy than IL-10.

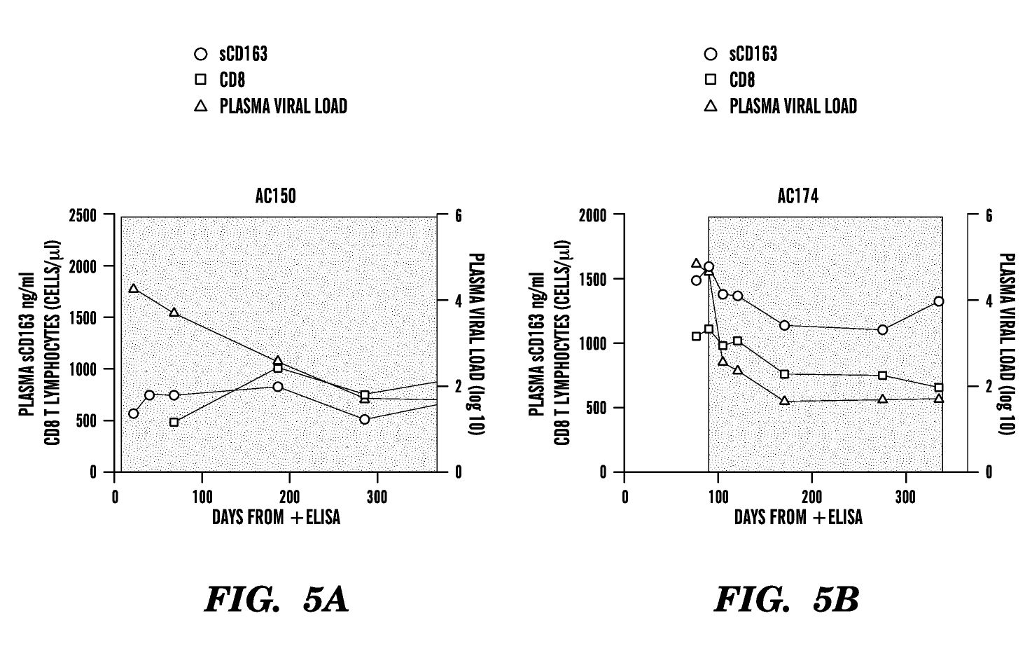

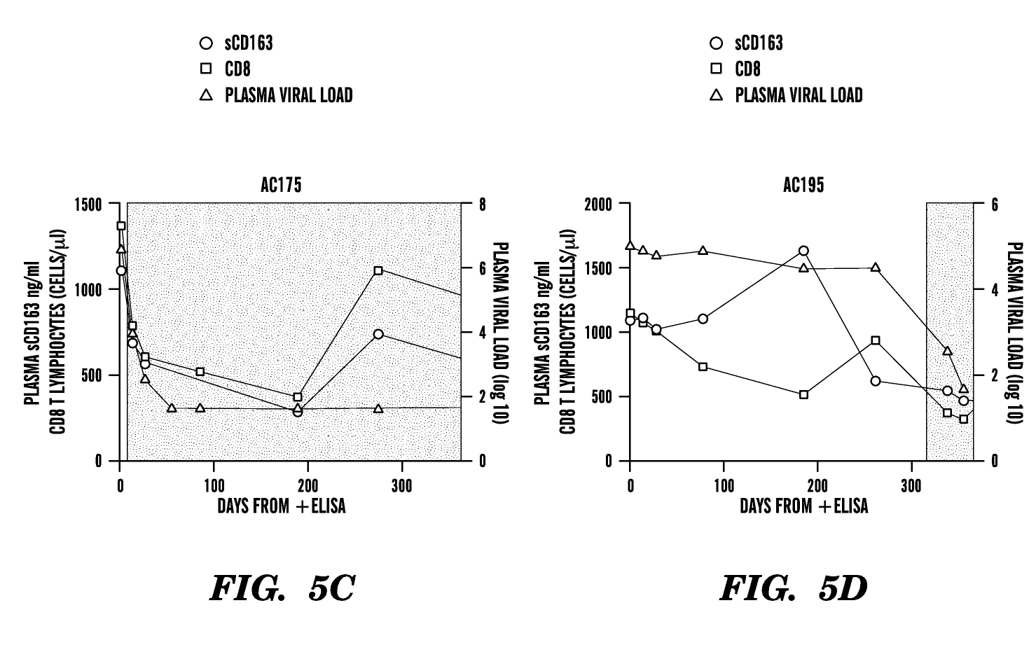

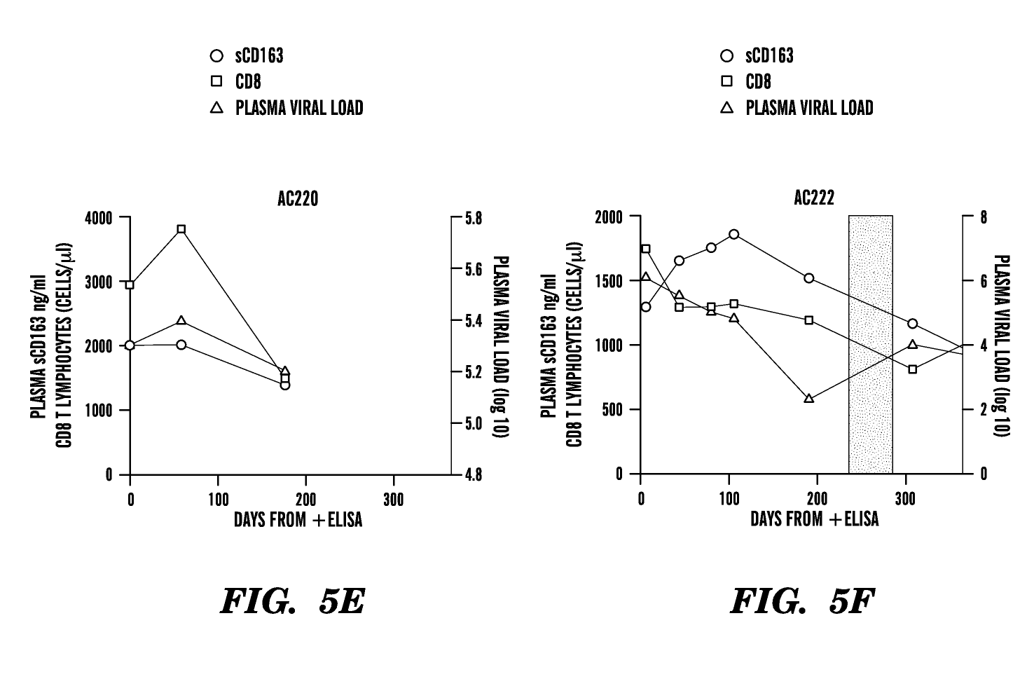

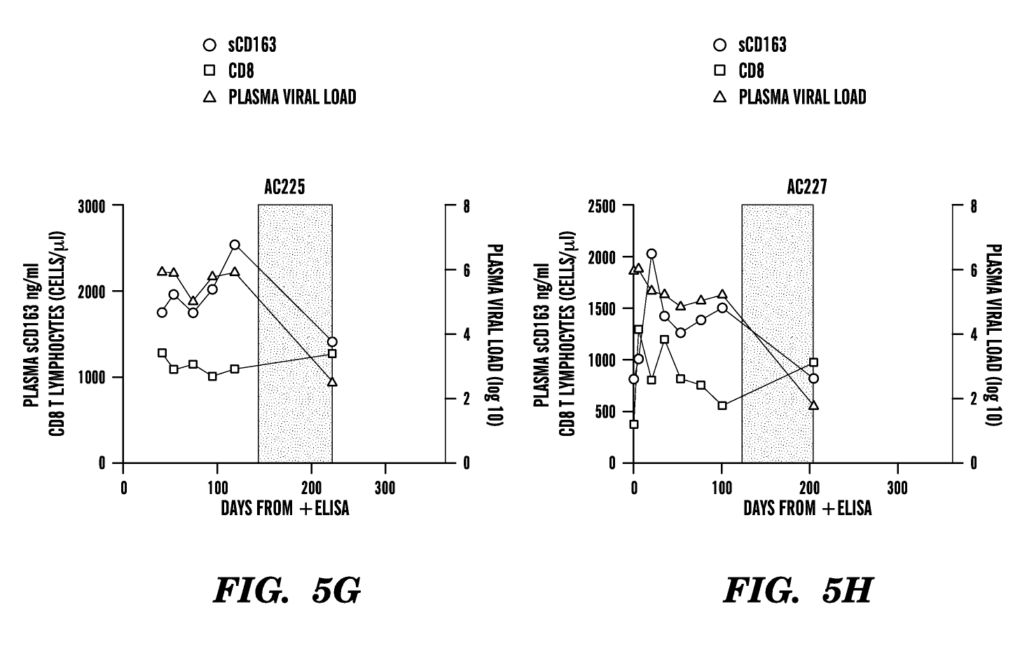

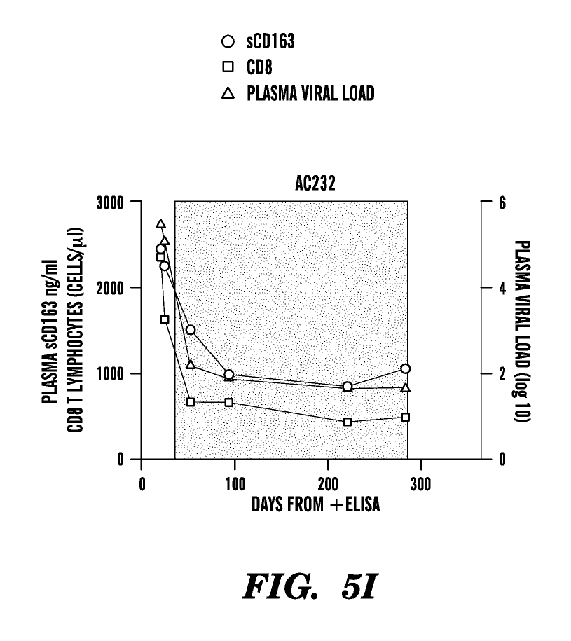

In light of data obtained from early HIV-infected individuals before and after 3 months of ART, an additional cohort of nine early HIV-infected subjects were examined over the entire first year after seroconversion. Longitudinal analyses of sCD163, plasma viral load, absolute numbers of CD8+ and CD4+ T lymphocyte from plasma were performed (FIG. 5). sCD163 was elevated at seroconversion and was readily decreased following ART initiation (FIG. 5, gray areas). In these early HIV-infected subjects, the changes in plasma viral load did predict changes in sCD163 (P=0.03), but therapy use did not influence the change in sCD163 (P=0.35) (Table 2). There was no significant difference between individual subjects (P=0.28) or between therapy status groups (P=0.35) (Table 2). As a result, there is evidence of a common linear relationship between changes in both plasma viral load and sCD163 across all subjects and therapy status groups (positive relationship, R2=0.19, P<0.0002). A relationship was seen between CD8+ counts and sCD163 (P<0.001), but it varied among subjects (P<0.0006) (Table 2). There was no significant relationship between absolute CD4+ T lymphocytes and sCD163 in these subjects, since the model using changes in absolute CD4+ T lymphocytes was not significant (Table 2).

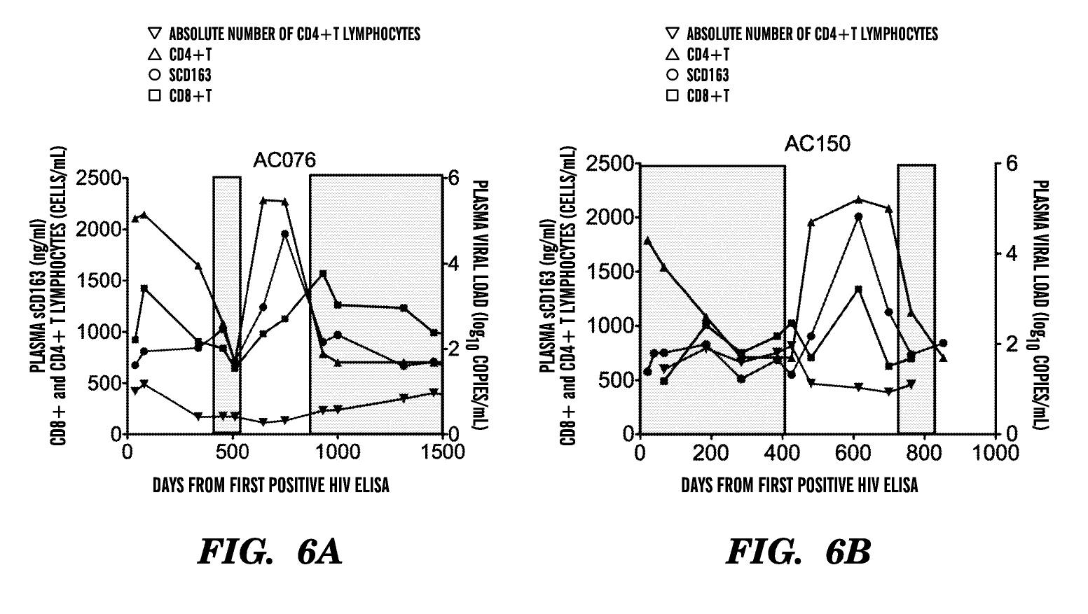

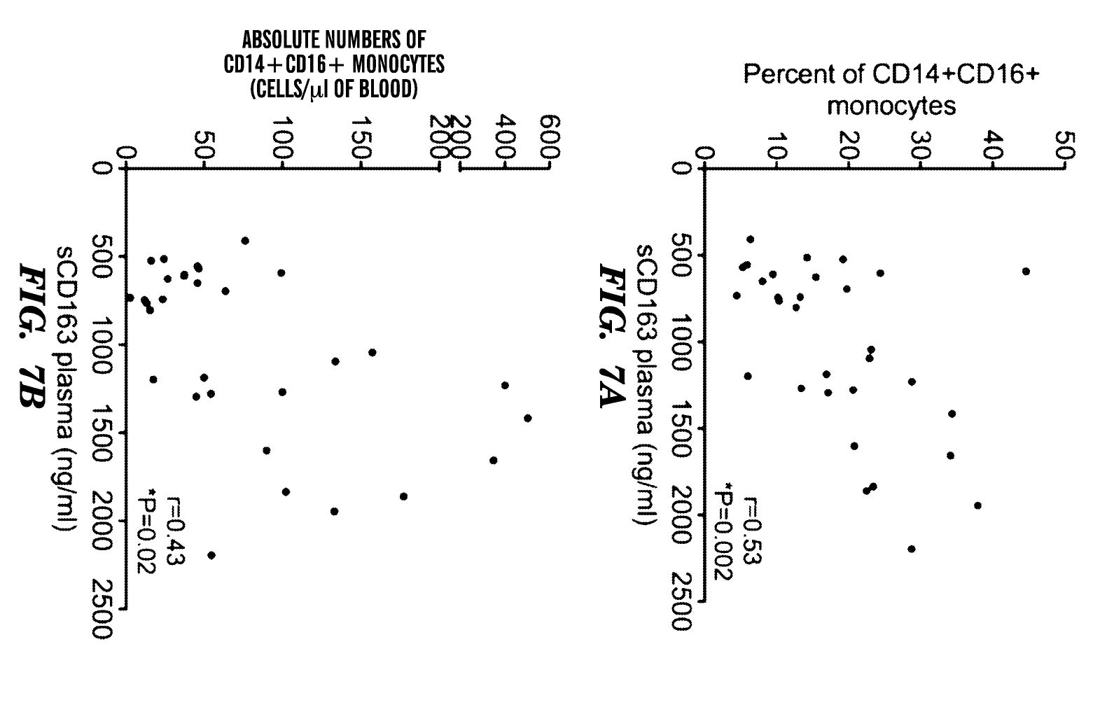

In light of data obtained from early HIV-infected individuals before and after 3 months of ART, we studied an additional cohort of nine early HIV-infected subjects examined the entire first year after seroconversion. Longitudinal analyses of sCD163, plasma virus, and absolute numbers of CD8+ and CD4+ T lymphocytes were performed. sCD163 was elevated at seroconversion and was readily decreased following ART initiation. Generalized linear model-repeated measures analyses were used to analyze relationships between sCD163, plasma virus and CD4+ and CD8+ T lymphocytes from serial samples in early HIV-infected subjects. Changes in plasma viral load predicted changes in sCD163 (P=0.03). A relationship was seen between sCD163 and CD8+ cells (P<0.001), but not CD4+ cells. Four early HIV-infected individuals underwent planned interruptions in therapy after the first year of infection. During interrupted therapy (FIG. 6A-6B, white area), plasma virus was detectable, followed by increased sCD163 and CD8+ T lymphocytes (FIG. 6A-6B). With re-initiation of ART, plasma virus, sCD163 and CD8+ T cells decreased to levels similar to those before ART interruption (FIG. 6A-6B, gray area). The number of CD4+ T lymphocytes was not altered (FIG. 6A-6B). These data underscore the role of macrophage activation and enhanced innate immunity during periods of interrupted therapy, which can be monitored by sCD163 in plasma.

TABLE 1
Subject Information

| | HIV− | Chronic HIV+ | Early HIV+ |
|---|---|---|---|
| Number of | 29 | 30 | 14 |
| subjects | | | |
| Age (Range) | 39 years | 39 years | 39 years |
| | (26-57) | (26-57) | (22-51) |
| Number of Males | 25 males | 26 males | 14 males |
| (Percentage) | (86%) | (87%) | (100%) |
| Ethnicity | 4 blk, 6 his, | 5 blk, 5 his, | 2 blk, 12 wht |
| | 19 wht | 19 wht, 1 oth | |
| ^{A}Days on ART | NA | 97 days (75-116) | 87 days (55-120) |
| (Range) | | | |
| Plasma viral load | NA | 4.5 mlog10 ± 0.9 | 5.4 mlog10 ± 1.3 |
| (Pre-ART) | | | |
| Plasma viral load | NA | <2.6 mlog10 | <1.7 mlog10 |
| (3 mos ART) | | (undetectable) | (undetectable) |
| CD4 (Pre-ART) | ND | 242 cells/ul | 481 cells/ul |
| | | blood ± 178 | blood ± 258 |
| CD4 (3 mos ART) | ND | 369 cells/ul | 708 cells/ul |
| | | blood ± 199 | blood ± 272 |

^{A}Average number of days from initiation of therapy to 3 months ART plasma sample
B: Averages ± standard deviations are shown.
C: NA = not applicable, ND = not determined, blk = African American, his = Hispanic, wht = Caucasian, oth = other

TABLE 2
Predictors of Change in sCD163 in early HIV-infected subjects.

| | Change in | Change | Change |
|---|---|---|---|
| | plasma viral load | in CD8 | in CD4 |
| Overall model | 0.04 | 0.001 | 0.35 |
| Predictor | 0.03 | 0.95 | — |
| Predictor × subject | 0.28 | 0.0006 | — |
| Predictor × therapy status | 0.35 | 0.78 | — |

General linear models (GLM) were created to determine if the change in viral load, numbers of CD4+, or CD8+ T lymphocytes (Predictors) had the ability to predict the change in sCD163. Each model also attempted to determine if the relationship between each predictor and sCD163 differed for each subject (Predictor x subject) or if it differed depending on current therapy status (Predictor x therapy status). Differences in log plasma viral load, CD4+ and CD8+ numbers was calculated for each time point to obtain sequential differences for each variable. Sequential data for all nine acutely infected subjects were pooled. Variable distributions in GLMs were assumed to be normal. Individual effect terms were only reported if the overall model was significant (P<0.05).

We demonstrate that sCD163 in plasma returns to normal levels with ART in early patients but not in chronic patients. These data suggest that chronic macrophage activation, and potential dysfunction, is not an immediate outcome of HIV infection. Instead it appears to take (in this study) at least a year to attain chronic macrophage activation states, as defined by sCD163 elevation, which is incompletely responsive to suppressive ART.

Data are provided herein that show that plasma sCD163, a direct product of monocyte/macrophage activation, is substantially increased during early and chronic HIV infection, and decreased with successful ART. Moreover, with successful ART in early HIV infection sCD163 levels returned to basal HIV-seronegative levels, in contrast to successful ART in chronic HIV-infected individuals where sCD163 levels were still elevated. sCD163 is considered to be an important anti-inflammatory molecule and herein its potential as a sensitive plasma marker for active HIV infection and innate immunity during AIDS is demonstrated. It has been previously demonstrated that expansion of monocyte/macrophage from bone marrow correlated with rapid onset of SIV-AIDS and CNS neuropathology (Hasegawa, A., et al., 2009. Blood; Burdo, T. H., et al., 2010. PLoS Pathog 6:e1000842), where sCD163 in plasma correlated with the magnitude of blood monocyte BrdU uptake.

Only one case report has previously described surface CD163 associated with acute HIV infection, although they did not directly measure plasma sCD163 levels (Delanghe, J. R., et al., Clin Chem Acta 411:521-523). This report described a patient with probable impaired hemoglobin scavenging due to a transient CD163 pathway impairment following an acute HIV infection (Delanghe, J. R., et al., Clin Chem Acta 411:521-523). At the time of evaluation, the patient had low levels of CD163 on monocytes and accumulation of haptoglobin:hemoglobin in the serum. The patient improved clinically and biochemically after initiation of ART suggesting that the syndrome resulted from a transient CD163 impairment that was HIV-mediated (Delanghe, J. R., et al., Clin Chem Acta 411:521-523).

To date, two reports have described CD163 shedding associated with HIV infection (Knudsen T B, et al., Clin Microbiol Infect 2005; 11:730-5; Delanghe J R, et al., Clin Chim Acta 2010; 411:521-3). One case report described a patient with impaired hemoglobin scavenging following acute HIV infection (Delanghe J R, et al., Clin Chim Acta 2010; 411:521-3). The patient had low levels of CD163 on monocytes and accumulation of haptoglobin:hemoglobin in the serum, but improved with ART suggesting a transient CD163 impairment that was HIV-mediated (Delanghe J R, et al., Clin Chim Acta 2010; 411:521-3). The second report showed sCD163 levels in serum predicted survival in tuberculosis (TB) patients where those with HIV and TB had the highest levels of sCD163 (Knudsen T B, et al., Clin Microbiol Infect 2005). The relationship between HIV, monocyte/macrophages and sCD163 with disease was less clear in this study due to TB co-infection (Knudsen T B, et al., Clin Microbiol Infect 2005).

Herein we demonstrate that plasma sCD163 are elevated shortly after seroconversion, and return to normal levels with early ART. It was only with ART early in HIV infection that sCD163 levels normalized. Soluble CD163 levels in subjects with chronic HIV infection did not revert to normal after 3 months of ART regimens. These data indicate that chronic macrophage activation and potential dysfunction is not an immediate outcome of HIV infection; rather it takes (in this study) at least a year to attain macrophage activation states as defined by sCD163 elevation, that are incompletely responsive to suppressive ART.

It is noted that in clinical practice ART is frequently unsuccessful and data for sCD163 levels with unsuccessful ART is not shown. Based on our results, we believe that with unsuccessful ART sCD163 would remain elevated. Changes in sCD163 could be a direct effect of antiretrovirals themselves as they may have a direct effect on macrophages, although without wishing to be bound by theory it is more likely that they act indirectly through reducing immune activation related to reduced viral replication. Of the standard antiretrovirals in clinical use, few are directly immune suppressive of macrophages or T lymphocytes (Lova, L., et al., 2005. Aids 19:137-144; Perno, C. F., et al., 2006. Antiviral Res 71:293-300) and thus unlikely to explain the sCD163 modulation effects described.

We found a significant correlation between sCD163 in plasma and absolute number of monocytes (using an automated differential) in HIV infected subjects before ART. This correlation was not maintained after ART. In clinical leukocyte differentials, one can sometimes see a relative cytopenia (e.g., monocytopenia) when a population of cells is expanding. People starting effective ART likely have relatively rapid expansion of lymphocytes, which may cause the relative monocyte proportion to decline, potentially explaining the reason for the correlation before starting ART and the absence of correlation afterward. Additionally, a direct correlation between plasma sCD163 and expansion of specific monocyte subsets is not ruled out. In fact, with SIV infection, a significant correlation between plasma sCD163 and the percent and absolute number of CD14+ CD16+ monocytes was observed (FIGS. 8A and 8B).

Expansion of the number and/or relative percentage of CD14+ CD16+ monocytes have been shown to correspond with disease progression and macrophage-mediated histopathology in AIDS, including HIVE (human immunodeficiency virus encephalitis) and SIVE (simian immunodeficiency virus encephalitis) (Pulliam, L., et al., 1997. Lancet 349:692-695; Fischer-Smith, T., et al., 2001. J Neurovirol 7:528-541; Thieblemont, N., et al., 1995. Eur JImmunol 25:3418-3424). Similarly, the inventors have shown a biphasic increase in the percentage and absolute number of CD14+ CD16+ monocytes that occurs first with peak SIV viremia, and then later with AIDS and SIVE (Williams, K. C., and Hickey, W. F. 2002. Annu Rev Neurosci 25:537-562, Williams, K., et al., 2005. J Clin Invest 115:2534-2545; Williams, K. C., et al., 2001. JExp Med 193:905-915; Williams, K., et al., 2002. Am J Pathol 161:575-585). Interestingly, this biphasic pattern in the number of activated monocytes occurs regardless of whether animals are rapid or conventional progressors. Because a biphasic increase of sCD163 in these animals and then decrease was not found but only a biphasic increase and then decrease in monocyte populations was found, without wishing to be bound by a theory, it is possible that sCD163 found in plasma also comes from activated tissue macrophages (Burdo, T. H., et al., 2010. PLoS Pathog 6:e1000842). Data from the inventor's laboratory and others show that CD14+ CD16+ monocytes and then CD14+ cells have the highest levels of CD163 surface expression, with CD14+ CD16− having little to none (Buechler, C., et al., 2000. J Leukoc Biol 67:97-103). Without wishing to be bound by a theory, this appears to indicate that sCD163 shed during HIV infection is from CD14+ CD16+ monocytes.

It has been shown that following 48 weeks of ART, LPS plasma levels were significantly decreased, indicating that a reduction in HIV replication is associated with the restoration of mucosal immune protection of the gut (Brenchley, J. M., et al., 2006. Nat Med 12:1365-1371). Additionally, Ancuta et al. showed that LPS levels were elevated after less than 8 weeks of ART, as compared longer than 8 weeks of ART (Ancuta, P., et al. 2008. PLoS ONE 3:e2516). We show data that indicate LPS levels after 3 months of ART were not significantly changed in the cohort of chronic HIV-infected subjects examined indicating that restoration of the gut might take longer than 3 months post treatment. In this report, individuals were examined after only 3 months of ART but the effect of long-term ART on LPS or other serum markers was not examined. The effect of long-term viral suppression on plasma sCD163 and chronic monocyte/macrophage activation was also not examined. We demonstrate that sCD163 and IL-10, but not others, decline with effective ART. Macrophages, as well as dendritic cells, neutrophils, and B and T lymphocytes, produce IL-10 (Saraiva, M., and O'Garra, A. 2010. Nat Rev Immunol 10:170-181), whereas sCD163 is solely shed from monocyte/macrophages (Droste, A., et al., 1999. Biochem BiophysRes Commun 256:110-113). Thus, plasma sCD163, more so than IL-10, is useful in examining persistent innate immune activation by macrophages with HIV infection in ART-treated individuals.

HIV-infected monocytes are found in individuals on ART and proviral DNA can be measured in monocytes despite undetectable plasma viral loads (Sonza, S., et al., 2001. Aids 15:17-22; Harrold, S. M., et al., 2002. AIDS Res Hum Retroviruses 18:427-434; Zhu, T., et al., 2002. J Virol 76:707-7 16). Reverse transcriptase inhibitors although effective on early-infected monocyte/macrophages, were not effective on chronically infected monocyte/macrophages (Perno, C. F., et al., 2006. Antiviral Res 71:293-300). Protease inhibitors were the only drugs active in chronically infected monocyte/macrophages (Perno, C. F., et al., 2006. Antiviral Res 71:293-300).

These data, along with the observation that sCD163 levels returned to HIV-seronegative levels with successful ART in early HIV-infected individuals, provide evidence that early ART may dampen the inappropriately activated immune system. When treating chronically infected individuals there is residual macrophage activation after 3 months, as seen with sCD163 levels higher than seronegative controls. This indicates that sCD163 is a biomarker of HIV-disease activity in longer-lived cells, such as macrophages.

In subjects with chronic HIV infection, sCD163 can be used as a plasma marker for monitoring macrophage activation and response to ART even when plasma viral load is undetectable. However, the cost-effectiveness of this biomarker in the clinic is not yet known. The exact function of sCD163 is unknown, but is thought to be involved in recycling extracellular iron, thus inhibiting growth of pathogens, especially bacteria that require iron for survival (Weaver, L. K., et al., 2006. J Leukoc Biol 80:26-35; Madsen, M., et al., 2004. J Biol Chem 279:51561-51567). sCD163 also has direct anti-inflammatory effects as it has been shown to inhibit T lymphocyte activation and proliferation (Hogger, P., and Sorg, C. 2001. Biochem Biophys Res Commun 288:841-843). This T cell regulatory role for sCD163 may indirectly indicate changes in HIV pathogenesis in vivo. Early in HIV infection, HIV is generally macrophage-tropic (M-tropic) and effective ART decreases sCD163 to normal levels. As a natural inhibitor of T cell activation, sCD163 increases may be the M2 anti-inflammatory macrophage attempt to limit viral replication/spread through dampening of T cell activation states. Later in disease, the sCD163 response is less effective and with ART's inability to restore sCD163 to normal levels may signify a switch from M-tropic to T-cell tropic (T-tropic) HIV. The enhanced sCD163 in the chronic HIV-infected individuals may signify the presence of a chronic macrophage activation state attempting to control T-tropic HIV infection through inhibiting T cell activation and proliferation. The exact function of sCD163 is unknown, but it is involved in recycling extracellular iron and thus inhibiting growth of bacterial pathogens. Additionally, sCD163 has been shown to directly inhibit T lymphocyte activation and proliferation. Early in HIV infection, HIV is generally macrophage-tropic (M-tropic) and effective ART decreases sCD163 to levels similar to controls. As a natural inhibitor of T cell activation, sCD163 increases may represent an attempt of anti-inflammatory macrophages to limit viral replication and spread. Later in disease, the sCD163 response is less effective and with ART's inability to restore sCD163 to normal levels may signify a switch from M-tropic to T-cell tropic HIV. The enhanced sCD163 in the chronic HIV-infected individuals may signify the presence of a chronic macrophage activation state attempting to control T-tropic HIV infection through inhibiting T-cell activation and proliferation.

Here, the presence of protein marker sCD163 was identified; sCD163 is only made by monocyte/macrophages and is linked to monocyte expansion. Since monocyte expansion predicts progression to AIDS, sCD163 will be useful in predicting HIV disease progression. This is the first observation within HIV-infected individuals of a marker in plasma that is both exclusive to monocytes and a marker of activation of the innate immune system. Altogether, these findings underscore the role of monocytes and innate immunity in HIV pathogenesis. Here, we have identified the presence of protein marker, sCD163 made by monocyte/macrophages and linked to monocyte expansion. Since monocyte expansion predicts progression to AIDS, we proposed that sCD163 is useful in predicting HIV disease progression. This is the first observation within HIV-infected individuals of a marker in plasma that is both exclusive to monocytes and a marker of activation of the innate immune system that parallels HIV replication. These results underscore the significance of innate immune responses by monocyte/macrophages in HIV pathogenesis. This work points to the utility of sCD163 as a marker of HIV activity for AIDS therapies targeting HIV replication, monocyte/macrophage immune activation and pathogenesis.

Health care providers usually withhold ART until CD4 levels decline below 350 CD4 cells. This study suggests that waiting until such thresholds are reached may be detrimental, since ART initiation in early-infected subjects normalized monocyte activation unlike in chronically infected patients. Such irreversible monocyte activation can have adverse consequences in HIV, including insulin resistance, lipodystrophy and vascular complications. We note that in clinical practice ART is frequently unsuccessful. Our data would predict that with unsuccessful ART, sCD163 would remain elevated. It is possible that changes in sCD163 we find could be from a direct effect of antiretrovirals on macrophages, although we believe it is more likely that they act indirectly through reduced viral replication. Of the standard antiretrovirals in clinical use, few have demonstrated direct immune suppressive effects on macrophages or T lymphocytes (Lova L, et al., AIDS 2005; 19:137-44; Perno C F, et al., Antiviral Res 2006; 71:293-300) and thus are unlikely to account for the sCD163 modulation effects we describe.

### Exemplary Material and Methods:

The patient samples used in this study were obtained from ongoing studies approved by the University of California at San Diego's or Massachusetts General Hospital's Institutional Review Board. All study participants and/or legal guardians provided written informed consent.

Chronically HIV-Infected and HIV-Seronegative Individuals:

Human plasma samples from chronically HIV+ and HIV-seronegative individuals were collected. EDTA—anticoagulated plasma was obtained from 30 chronically HIV-infected (>1 year infected) individuals at 2 times during infection (see Table 1 for clinical information): 1) when they had high viral load before starting a new regimen (Chronic HIV+Pre-ART), and 2) 3 months after starting a new regimen when viral load was undetectable (Chronic HIV+3mos Post-ART). At time point 1, fourteen of the subjects were ART naive, while others had previously taken a different regimen. Plasma was also obtained from 29 age-matched HIV-seronegative individuals (HIV-). This study was approved by the University of California at San Diego's Institutional Review Board. All subjects provided informed consent for all study procedures. Hematological parameters were measured at the UCSD Medical Center clinical laboratory.

Early HIV-Infected Subjects:

Three cohorts of early HIV-infected subjects were examined. Early HIV-infection herein is defined as the first year after seroconversion (as defined by the appearance of HIV-specific antibodies measured by an indeterminate or positive Western blot) (Kassutto, S., and Rosenberg, E. S. 2004. Clin Infect Dis 38: 1447-1453; Alter, G., et al., 2007. J Infect Dis 195:1452-1460; Lentz, M. R., et al., 2009. Neurology 72:1465-1472). All early HIV-infected subjects examined were identified during acute HIV infection (days-weeks from exposure). Three cohorts of early HIV-infected subjects were examined. For direct comparison to the chronic subjects, cohort 1 consisted of 14 early HIV-infected subjects in which plasma from EDTA-coagulated blood was available and examined at 2 time points (see Table 1 for clinical information): 1) naive to ART (Early HIV+Pre-ART), and 2) after 3 months of ART (Early HIV+3mos ART). The cohort 2 consisted of nine early HIV-infected subjects that were examined 1 year after seroconversion, eight of whom eventually went on ART. Cohort 3 consisted of four early HIV-infected subjects, who underwent planned therapy interruptions. The Massachusetts General Hospital's Institutional Review Board approved this study. All subjects provided informed consent. Hematological parameters were measured at Massachusetts General Hospital Clinical Microbiology laboratory.

Peripheral Blood Mononuclear Cells (PBMC):

PBMC were only available from 8 of the chronically HIV-infected subjects Pre-ART and 9 of the chronically HIV-infected subjects 3mos ART. Of these, two were matched pairs of Pre-ART and 3mos ART. PBMC were obtained from all 14 early HIV-infected subjects (cohort 1), both Pre-ART and after 3mos with ART. All PBMC were obtained at the same time as plasma used to measure sCD163.

Flow Cytometry:

Frozen PBMC were quickly thawed in a 37° C. water bath and washed twice in 40 mis of heated RPMI 20% FBS. Two million PBMC were incubated for 15 minutes with an antibody cocktail of CD14-Pacific blue, CD16-PECy7, HLA-DR-APCCy7, CD163-PerCpCy5.5, CD20-APC, CD3-Alexa Fluor 700, CD38-PE, CD4-FITC and CD8-Qdot 655. A Live/Dead stain kit was used to exclude non-viable cells. Samples were washed and fixed in 1% PFA and immediately acquired on a BD FACS Aria.

HIV RNA Quantitation:

Different assays for plasma viral load were used in early and chronic HIV-infected subjects. In the chronic HIV-infected individuals, plasma viral load was obtained using either ultrasensitive (undetectable=50 (1.7 log₁₀) copies RNA/ml of plasma) or standard assay (undetectable=400 (2.6 log₁₀) copies RNA/ml of plasma) (Amplicor Monitor, Roche, Indianapolis, Ind.). Therefore, for all chronic HIV-infected individuals, all undetectable levels were identified as 2.6 log₁₀ copies RNA/ml of plasma. In the early HIV-infected subjects, plasma viral load was obtained using TaqMan quantitative PCR with a lower limit of 48 (1.7 log₁₀) copies RNA/ml of plasma.

Hematological Parameters:

In the chronic HIV-infected subjects, absolute number of CD4+ T lymphocytes, platelets and monocytes, hematocrit and hemoglobin were obtained contemporaneously and at the UCSD Medical Center clinical laboratory. In early HIV-infected individuals, absolute CD4+ and CD8+ T lymphocytes numbers were measured by the Massachusetts General Hospital clinical Microbiology laboratory at the same visit as plasma obtained for sCD163. sCD163, OPN, sCD14, IL-6 ELISAs and LAL assay: Soluble CD163 (sCD163), osteopontin (OPN), IL-1 0, sCD14 and IL-6 in plasma were quantified by ELISA according to manufacturer's protocol (Trillium Diagnostics (sCD163), IBL-America (OPN) and R&D Systems (IL-10, sCD14 and IL-6)). The Diazo-coupled Limulus amebocyte lysate (LAL) assay (Associates of Cape Cod Inc.) was used to quantify endotoxin/liposaccharide (LPS) levels in plasma from SIV-infected animals, according to the manufacturer's protocol and as previously described (Burdo, T. H., et al., 2010. PLoS Pathog 6:e1000842).

Statistical Analysis:

For statistical analyses the Prism version 5.0a (GraphPad Software, Inc., San Diego, Calif.) software or Microsoft Excel version 12.2.3 and 12.2.4 were used. An ANOVA was first performed for analysis of variation among groups of data. If the ANOVA was significant (P<0.05), then post-hoc t-tests were performed. Paired t-tests were used for all matched samples. A Spearman rank test was used for all correlations

Generalized Linear Model (GLM)-Repeated Measures Analyses:

GLM-repeated measures analyses were used to analyze relationships between sCD163, plasma virus and CD4+ and CD8+ T lymphocytes from serial samples in early HIV-infected subjects during the first year after seroconversion. For each pair of successive measures, the change in viral load, T cells and therapy status (N-N, Y-Y, Y-N, and N-Y) was determined. All groups had more than one sample with the exception of the Y-N group. The Y-N group was excluded from analyses due to the low sample size (n=1). GLMs were performed using the changes in the aforementioned immunological variables as predictors, subject identifier and therapy status change groups as covariates, and the cross product between the predictor and each of the covariates (Table 2). Variable distributions in GLMs were assumed to be normal. Individual effect terms were only reported if the overall model was significant (P<0.05).

### Example 2

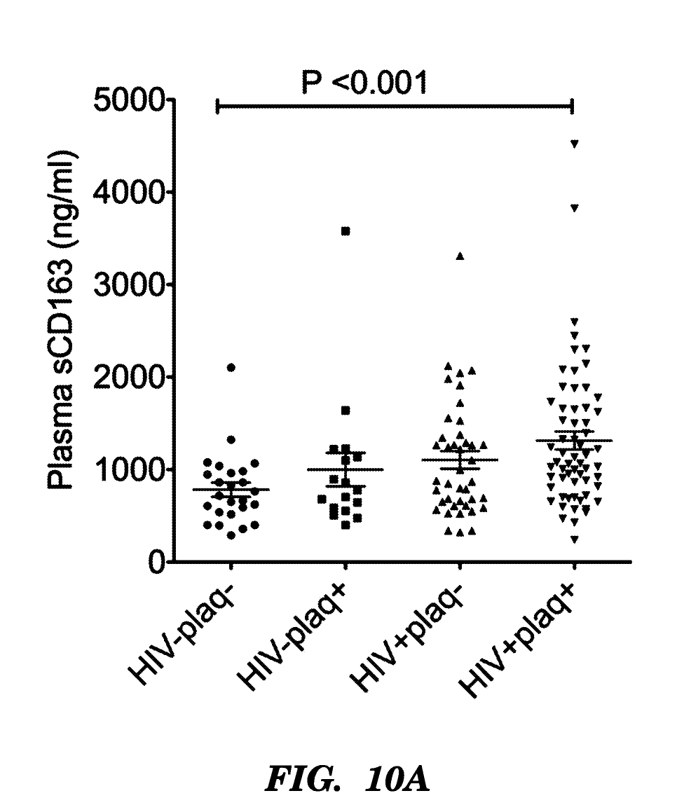

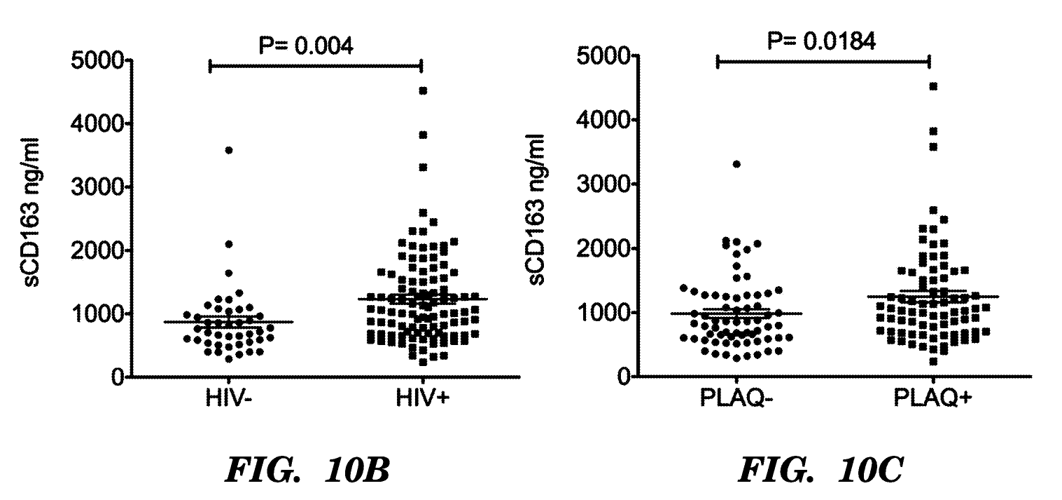

Along with the traditional cardiovascular risk factors (including diabetes, smoking, hypertension, body mass and high serum cholesterol), HIV-infected individuals are predisposed to increased risk due to immune activation, chronic inflammation and viral factors. Here, 146 male subjects were examined (104 HIV-infected individuals and 42 HIV-seronegative) with similar coronary artery disease (CAD) risk factors, without history or symptoms of CAD. The HIV-infected men had a higher prevalence of coronary atherosclerosis (59 vs. 39%) and higher coronary plaque volume compared to HIV seronegative men (Lo et al 2010, AIDS). As atherosclerosis involves infiltration by monocyte-derived macrophages and is a macrophage-mediated disease, sCD163, which is shed solely by activated macrophages, as a marker of coronary atherosclerosis in HIV-infected subjects, was investigated (see e.g., FIG. 10A-10C). Plasma sCD163 was significantly elevated in HIV-infected individuals and in subjects with coronary plaques present. The HIV-infected patients with the presence of plaques had the highest levels of plasma CD163, with HIV seronegative subjects without plaques with the lowest levels. Specifically, sCD163 levels highly correlated with the presence of non-calcified plaques, which are sites of active vascular inflammation. The monocyte-specific marker sCD163 is a novel plasma marker of coronary atherosclerotic in HIV-infected subjects. These results underscore the importance of activated macrophages and immune activation in coronary artery disease in HIV-infected patients.

As we showed, pro-inflammatory monocytes/macrophages contribute to increased atherosclerosis in HIV patients. We showed for the first time sCD163 in relationship to atherosclerotic plaque in HIV-infected patients.

102 HIV-infected and 41 HIV-seronegative men with equivalent cardiovascular risk factors and without history of coronary artery disease were prospectively recruited and underwent CT coronary angiography.

sCD163 levels and presence of plaque were significantly higher among ART-treated subjects with undetectable HIV RNA levels compared to seronegative controls (1172±646 vs. 883±561 ng/mL; p=0.02 for sCD163 and 61% vs. 39%, p=0.03 for presence of plaque). After adjusting for age, race, lipids, blood pressure, glucose, smoking and HIV, sCD163 remained independently associated with noncalcified plaque (p=0.04). Among HIV patients, sCD163 was associated with the natural log of coronary segments with noncalcified plaque (r=0.22, p=0.01), but not with calcium score. In contrast, markers of generalized inflammation including CRP, and D-dimer were not associated with sCD163 or plaque among HIV-infected patients.

We showed that sCD163, a monocyte/macrophage activation marker, is increased in association with noncalcified coronary plaque in men with chronic HIV infection and low or undetectable viremia. These data show important role of chronic monocyte/macrophage activation in the development of noncalcified vulnerable plaque.

We had observed that patients with HIV infection have a higher prevalence of subclinical coronary atherosclerosis and greater burden of coronary atherosclerotic plaque, particularly noncalcified plaque, than HIV-seronegative subjects with similar cardiovascular risk factors, even when they are young, with low Framingham risk score and free of symptoms of cardiovascular disease1. The plaque characteristics in this young asymptomatic HIV patient population were more likely to be noncalcified, rather than calcified plaque lesions. Plaques with necrotic lipid rich cores composed of pro-inflammatory immune cells are more vulnerable to rupture and are often noncalcified with low attenuation on computerized tomography (CT) (Motoyama S, et al., J Am Coll Cardiol. 2007; 50:319-326; van der Wal A, Becker A, van der Loos C, and Das P, Circulation. 1994; 89:36-44).

Pro-inflammatory monocyte and macrophage subsets are an integral component of the atherosclerotic process and have been shown to migrate to atherosclerotic plaque (Swirski F K, et al. J Clin Invest. 2007; 117:195-205).

Hypothesizing that activation of the innate immune system, contributes to CVD, we investigated markers of macrophage activation in association with atherosclerotic disease among HIV-infected patients, with a particular focus on those with undetectable viral load. CD163, a scavenger receptor specifically expressed on the surface of macrophages and monocytes, is involved in the uptake of hemoglobin-haptoglobin and mediates the clearance of hemoglobin (Fabriek B O, et al., Immunobiology. 2005; 210:153-160). Soluble CD163 (sCD163) is shed via proteolytic cleavage at the cell surface to a greater extent by activated monocytes/macrophages and is found in plasma. We have previously demonstrated that plasma sCD163 levels correlated with monocyte expansion from bone marrow, the rate of AIDS progression and severity of macrophage-mediated AIDS pathogenesis in SIV-infected rhesus macaques (Burdo T H, et al., PLoS Pathog. 2010; 6:e1000842). sCD163 was also recently shown to be a novel marker of HIV disease activity in early infected and chronically infected patients (Burdo and Williams, J Infect Dis. 2011 July; 204(1):154-63).

Levels of sCD163 are increased in non HIV-infected patients with angiographically significant coronary artery disease and related to the extent of coronary artery disease (Aristoteli L P, et al., Atherosclerosis. 2006; 184:342-347), but the relationship of sCD163 to atherosclerosis has not been studied specifically among HIV-infected patients in whom activation of the innate immune system is an important process. In this study, we investigated the relationship of activated monocyte/macrophages, as measured by sCD163 in plasma as well as other markers of monocyte/macrophage activation using flow cytometry, to atherosclerotic plaque and plaque characteristics in HIV-infected patients.

Study Participants:

One hundred and forty-three men participated in the current study. One hundred and two men with HIV infection were prospectively recruited from HIV clinics and community health centers in the Boston area as well as by newspaper advertisements. Forty-one HIV-seronegative control men were simultaneously prospectively recruited from the same communities and chosen to have similar cardiovascular risk factor profile to the HIV group. Family members, partners and friends of the HIV patients were especially encouraged to participate as controls in order to ensure similar characteristics between the two groups. Other than HIV disease, inclusion and exclusion criteria were identical for both groups. Participants aged 18-58 years, without known cardiac disease or symptoms suggestive of cardiac disease (any current or prior heart disease, including angina, arrhythmias, valvular heart disease, pericarditis, congestive heart failure, or any prior treatment for coronary artery disease (CAD) or any heart disease) were recruited. Participants underwent cardiac CT imaging only as part of the study protocol and not for clinical indications. HIV and control participants were not recruited with regard to any changes in body composition, weight, or metabolic criteria. HIV and control participants with known renal disease or creatinine more than 1.5 mg/dl or estimated creatinine clearance less than 70 ml/min were excluded to minimize risk of contrast nephropathy. HIV-infected patients receiving combination anti-retroviral therapy (ART) at the time of the study were required to be on stable therapy for more than 3 months. All participants provided informed consent. This study was approved by the institutional review boards of Massachusetts General Hospital and Massachusetts Institute of Technology. Cardiac CT data were previously published in a subset of the patients (Lo J, et al., Increased prevalence of subclinical coronary atherosclerosis detected by coronary computed tomography angiography in hiv-infected men. AIDS. 2010; 24:243-253). We have continued to expand this cohort and now report novel data not previously published on monocyte activation using sCD163, flow cytometry, and other markers, including LPS, sCD14, osteopontin, hsIL-6, and D-dimer.

Study Procedures and Assessment of Cardiovascular Risk Factors:

Data on sociodemographic factors, cardiovascular risk factors, medical history, family history, behavior, including smoking, medications and prior nadir CD4 count were obtained. For diabetes mellitus, both clinical history and prevalence based on fasting glucose were determined. All participants fasted at least 12 h before blood draws. Framingham risk score was calculated (Wilson P W F, et al. Circulation. 1998; 97:1837-1847). Cardiac multidetector row computed tomography and computed tomography angiography imaging were performed using a 64-slice CT scanner (Sensation 64; Siemens Medical Solutions). For full description, see Lo et al. AIDS. 2010; 24:243-253. Assessment of coronary atherosclerotic plaque burden and stenosis were determined by a consensus reading between two investigators, including a cardiologist and a radiologist with significant experience in the interpretation of cardiac CT. The presence of any coronary atherosclerotic plaque, whether calcified or noncalcified was determined. Discrete coronary segments affected by plaque were counted, and these segments were characterized as to whether they were noncalcified. The number of noncalcified segments per patient was determined. The percentage of noncalcified plaque was also determined, by dividing the number of noncalcified segments by the total number of segments affected with any plaque, per subject. Only those subjects with plaque could be included in this analysis. Presence of severe coronary artery stenosis was defined as luminal obstruction >70% diameter in any coronary segment. Physicians analyzing the scans were blinded to the participants' clinical history or HIV status. Agatston calcium score was calculated using the noncontrast CT images by standardized techniques10.

Inflammatory, Metabolic, Biochemical, and Immunologic Parameters:

Plasma sCD163, MCP-1, IL-6, sCD14 and osteopontin were quantified by ELISA according to manufacturer's protocol (Trillium Diagnostics (sCD163), R&D Systems (high sensitive IL-6, MCP-1 and sCD14) and IBL-America (osteopontin)). The endpoint LAL assay (Associates of Cape Cod) was used to measure LPS as previously described6. Total cholesterol, HDL, LDL, triglycerides, glucose and creatinine were determined using standard techniques. CRP was measured using ELISA. Quantitative determination of D-Dimer in plasma was performed by immuno-turbidometric method. CD4+ and CD8+ T cell counts were assessed by flow cytometry. HIV RNA levels was determined by ultrasensitive RT PCR (Roche Amplicor Monitor) (lower limit of detection=50 copies/mL). HIV testing was performed by ELISA (Abbott) and confirmed by Western blot. CMV IgG titers were measured using an enzyme-linked fluorescent immunoassay

Flow Cytometry:

Peripheral blood samples were collected in EDTA and prepared by standard methods and techniques previously established in the Flow Cytometry Laboratory at Massachusetts General Hospital (Cserti-Gazdewich C M, ET AL., Cytometry B Clin Cytom. 2009; 76:127-134; Cannizzo E, et al., Cytometry B Clin Cytom. 2010; 78:231-238). Eight fluorochrome conjugated antibodies were combined with 100 μL of whole blood, incubated for 15 minutes, then washed and fixed with 2 mL of 1×BD FACS™ Lysing solution (Beckton Dickinson, San Jose, Calif.) followed by 500 μL 1% paraformaldehyde (Sigma, St. Louis, Mo.). Samples were processed on a FACSCANTO™ II (Beckton Dickinson) and 50,000 events were collected and analyzed using FACSDIVA™ software. Data analysis was performed using FACSDIVA™ software with side light scatter versus CD14 gating used to identify monocytes and forward versus side light scatter gating used to identify lymphocytes. T lymphocytes were identified using populations of brightly stained CD4+ or CD8+ cells. HLA-DR and CD38 expression on CD4+ or CD8+ T cells was assessed by using negative lymphocyte populations to define negative controls.

Statistical Analysis:

Data are presented as mean±SD or median (interquartile range) depending on normality of distribution. Comparisons between two groups were performed using Student's T-test for normally distributed continuous variables and Wilcoxon rank sum test if the distribution was non-normal. Pearson correlation coefficients were used to assess correlations for normally distributed data. For non-normally distributed endpoints, data were either log-transformed or Spearman rho was used to assess correlation. Linear regression modeling was used in adjusted analyses for continuous outcome variables. Linear regression models were constructed with the natural log of nocalcified segments and % noncalcified plaque as the dependent variables of interest. Independent variables entered into the model included known traditional CVD risk markers (age, race, smoking, blood pressure, cholesterol, HDL, triglycerides, fasting glucose), in addition to markers of immune activation or inflammation that were significantly related on univariate regression analysis, including sCD163 and sCD14. A sensitivity analysis was performed including lipid lowering therapy in the model, because of the known effects of lipid lowering therapy to affect immune activation (Ganesan A, et al., J Infect Dis. 2011; 203:756-764). Two-tailed probability values are reported and statistical significance was assumed when p<0.05. All statistical analyses were performed using SAS JMP (SAS Institute).

Comparisons of Traditional Risk Factors and Markers of Monocyte/Macrophage Activation, Inflammation, and Atherosclerosis by HIV and Viral Load Status:

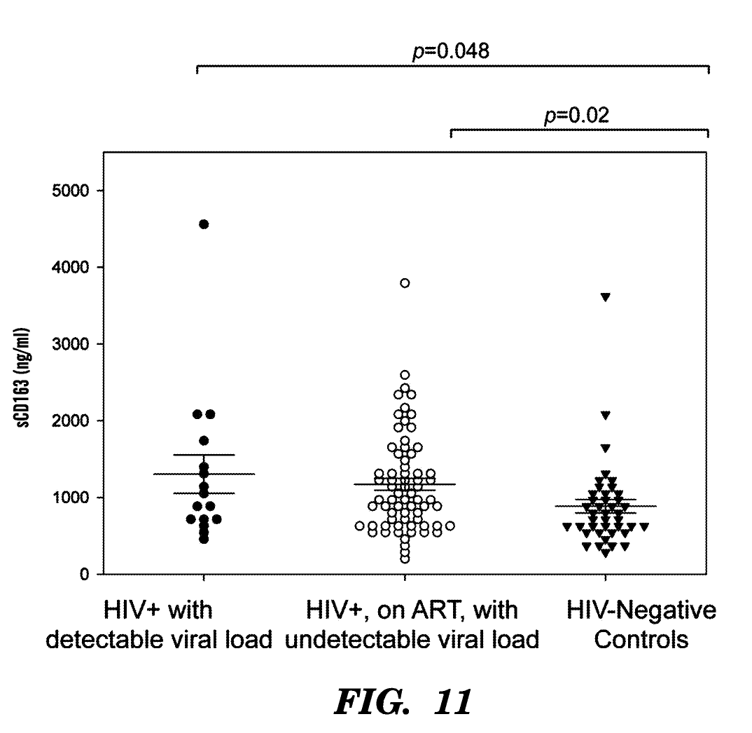

Demographic characteristics, overall Framingham risk score, prevalence of diabetes mellitus, and smoking were similar between HIV-infected subjects and seronegative controls (Table 3). Use of antihypertensive medications and lipid lowering therapy were higher in the HIV subjects. Viral load ranged from undetectable to 49,700 copies/mL. The majority (81%) of HIV-infected patients had undetectable viral load and all but 2 had viral load<15,000 copies. HIV-infected patients also had an increased prevalence of plaque and a greater number of coronary segments with noncalcified plaque on coronary computed tomography angiography than controls (Table 3). In contrast, the calcium score was similar between the groups. HIV-infected men demonstrated higher sCD163 in plasma than HIV-seronegative men (p=0.006) (FIG. 11). LPS, osteopontin, and hsIL-6 in plasma were higher in HIV patients than HIV-seronegative controls, while CRP and D-dimer did not differ (Table 3). The percentage of CD14+ CD16+ monocytes was higher in HIV patients than HIV-seronegative controls (Table 3).

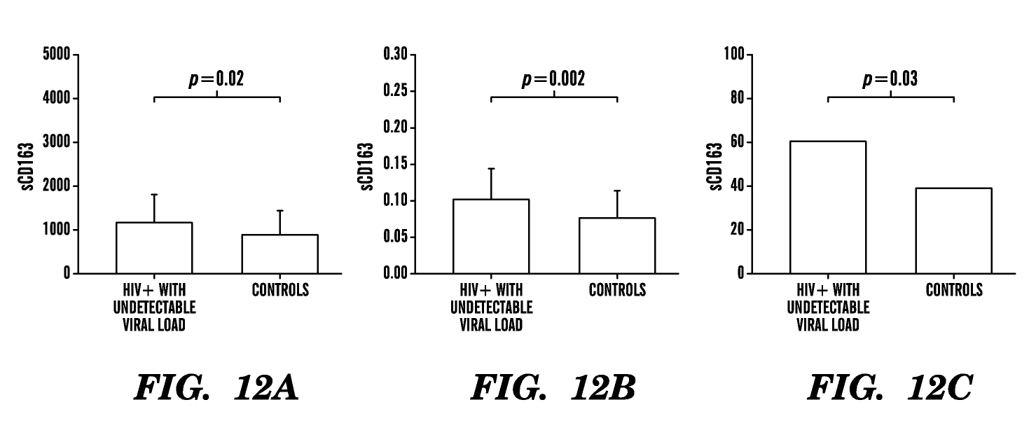

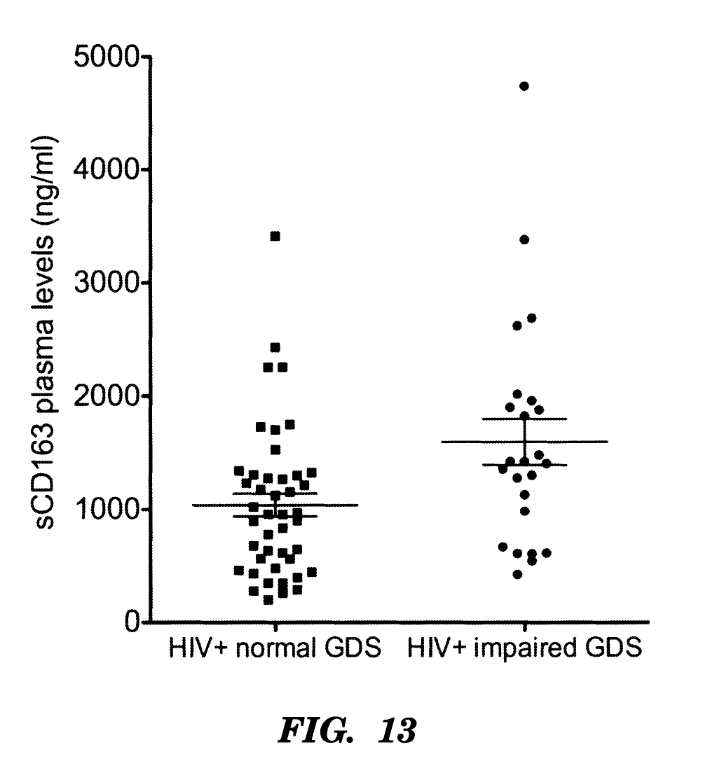

Among the subset of HIV-infected patients treated with ART and with undetectable HIV RNA levels, the presence of plaque, sCD163, LPS, osteopontin, percentage of CD14+ CD16+ monocytes again remained significantly higher in the HIV-infected patients compared to HIV seronegative control subjects (Table 3, FIGS. 12 and 13), while traditional risk factors, including smoking, diabetes, hypertension and overall Framingham risk score were again similar between the groups.

Relationship of sCD163 to Markers of HIV Disease Progression, Monocyte/Macrophage Activation, Generalized Inflammation, Immune Parameters and Plaque Characteristics, among HIV-Infected Patients:

sCD163 was associated with log HIV plasma viral load (r=0.22, p=0.04) and negatively correlated with CD4+/CD8+ ratio (r=−0.22, p=0.02) among all HIV infected patients (Table 4). sCD163 was not associated with nadir CD4 count (r=−0.13, p=0.24) nor current CD4 count (r=−0.12, p=0.23). sCD163 also correlated with LPS (r=0.27, p=0.007), sCD14 (rho=0.22, p=0.03), hs IL-6 (rho=0.27, p=0.01) and CMV IgG titres (rho=0.27, p=0.01). Furthermore, sCD163 was significantly associated with % CD14+ CD16+ monocytes (r=0.35, p=0.04) and with % HLA-DR+CD4+ T-cells (r=0.45, p=0.006) determined by flow cytometry.

Among HIV-infected patients on ART with undetectable HIV RNA levels, sCD163 was positively associated with natural log of coronary segments with noncalcified plaque (r=0.24, p=0.046), hs IL-6 (rho=0.32, p=0.01), and duration of HIV (r=0.23, p=0.05), and negatively associated with CD4+/CD8+ ratio (r=−0.29, p=0.02).

Relationship of sCD163 with Noncalcified Plaque in Unadjusted and Adjusted Analyses Among All Patients:

Among all patients, sCD163 was significantly associated with the natural log of the total number of noncalcified segments (r=0.22, p=0.01) (Table 4), the percentage of noncalcified plaque (rho=0.23. p=0.047), but not the natural log of calcium score (r=−0.04, p=0.62) (Table 5). In addition, sCD14 was significantly associated with the percentage of noncalcified plaque (rho=0.26, p=0.03). In contrast, markers of generalized inflammation such as CRP (rho=0.14, p=0.22) and hsIL-6 (rho=0.17, p=0.17), nor D-Dimer (rho=−0.12, p=0.29) were not associated with percent of noncalcified plaque. In contrast to the association seen between markers of monocyte/macrophage activation and noncalcified plaque, these markers were not significantly related to calcium score. Traditional cardiovascular markers were more strongly associated with calcium score, as opposed to noncalcified plaque, in contrast to monocyte/macrophage activation markers which were more strongly associated with noncalcified plaque (Table 5).

In multivariate regression analysis, after adjusting for HIV status, and traditional risk factors, as well as sCD14, sCD163 remained independently associated with the percentage of noncalcified plaque (p=0.02) and with number of segments with noncalcified plaque (p=0.008) among all subjects (Table 6A). Similarly sCD163 remained independently and significantly associated with percent noncalcified plaque (p=0.05) and segments with noncalcified segments (p=0.048), controlling for lipid lowering therapy in sensitivity analyses.

Subanalyses performed in HIV and non-HIV infected subjects, respectively, demonstrated that sCD163 remained significantly associated with noncalcified plaque segments in HIV (p=0.045) (Table 6B). No relationship was seen between sCD163 and noncalcified segments in non HIV patients (p=0.89) (Table 6C).

Data from this study support the hypothesis that monocyte and macrophage activation in HIV disease contributes to the formation of vulnerable atherosclerotic plaque. Specifically, we demonstrate that the monocyte/macrophage-specific marker, sCD163, is uniquely associated with increased noncalcified plaque among young, asymptomatic, HIV-infected men with low or undetectable levels of viremia and a long duration of HIV disease. sCD163 was significantly associated with increased noncalcified plaque burden, independent of traditional risk factors. These data lend insight into the dynamic process by which CVD develops among HIV-infected patients with low or undetectable plasma virus and suggest that macrophage activation, independent of traditional risk factors, contributes to a unique phenotype of inflammatory non-calcified coronary plaque among patients with long-term HIV infection.

In this study, we showed that traditional risk factors including age, Framingham risk score, cholesterol and LDL were significantly associated with increased calcium score, a measure of calcified plaque burden. In contrast, sCD163 and duration of known HIV infection were positively associated with non-calcified plaque. In addition, sCD14, another marker of monocyte activation, was associated with sCD163, and with noncalcified plaque in univariate analysis. However, sCD163 but not sCD14 remained significantly associated with plaque in the multivariate regression analysis also controlling for traditional risk factors. This was true in a combined analysis of all patients, controlling for HIV, and within the HIV subgroup alone. In contrast, the relationship between sCD163 and noncalcified plaque was not significant in multivariate regression among the non HIV patients.

In contrast to our data on sCD163, markers of generalized inflammation such as CRP did not show a relationship with plaque in our study, suggesting that among the large number of patients with controlled viremia, that monocyte/macrophage activation, and not generalized inflammation, contributes to increased plaque.

Taken together, these data are significant in that they demonstrate that specific types of atherosclerotic disease have different etiologies in the HIV population. Those patients with traditional risk factors resulting from dyslipidemia and insulin resistance, hypertension and other traditional risk factors will be expected to have more calcified lesions. However, we now show that even those with low traditional risk factors can have noncalcified disease and that this may be related to monocyte/macrophage activation, a point not previously appreciated in the HIV positive population. Plaques with necrotic cores are known to be more prone to rupture and histologically increased macrophage infiltration is seen in these necrotic cores (van der Wal A, Becker A, et al. Circulation. 1994; 89:36-44). Taken together, our data suggest a potential mechanism by which HIV infection may produce a phenotype of lipid-rich inflamed atherosclerotic plaques potentially via up-regulated monocyte activation and more vulnerable to rupture.

Inflammatory and immunologic factors may play important roles contributing to increased coronary artery disease in the HIV population as suggested by studies like SMART (El-Sadr W M, et al., N Engl J. Med. 2006; 355:2283-2296; Stein J H. N Engl J. Med. 2007; 356:1773-1775). Triant et al. and Lichtenstein et al. recently showed, among large cohorts, that acute myocardial infarction rates are related to low CD4 count, controlling for traditional risk factors and antiretroviral therapy (Triant V A, et al., J Clin Endocrinol Metab. 2007; 92:2506-2512; Lichtenstein K A, et al., Clin Infect Dis. 2010; 51:435-447). In addition, Kaplan et al. recently showed that HIV-associated T cell activation is related to subclinical carotid atherosclerosis (Kaplan R C et al., J Infect Dis., 2011 Feb. 15; 203(4):452-63. Epub 2011 Jan. 10). The current data show for the first time that monocyte/macrophage activation also contributes to subclinical atherosclerosis.

The current data extends the data from prior studies which showed endothelial dysfunction by brachial artery flow mediated vasodilation among those with longstanding low viremia (Hsue P Y, et al., AIDS. 2009; 23:2021-2027). We now demonstrate that among patients on stable antiretroviral therapy with undetectable viremia, monocyte activation is significant and relates most to noncalcified coronary plaque, which is increased among HIV patients. Atherosclerosis is an inflammatory process in which monocytes and T lymphocytes play important roles (Libby P. Inflammation in atherosclerosis. 2002; 420:868-874; Ross R., N Engl J. Med. 1999; 340:115-126). We and others have previously shown that CMV-induced immune responses may also be contributory to atherosclerosis in HIV patients (Lo J, et al., AIDS. 2010; 24:243-253; Melnick J L, et al., Jama. 1990; 263:2204-2207; Hsue P Y, et al., Aids. 2006; 20:2275-2283; Epstein S E, et al., Circulation. 2009; 119:3133-3141). In the current study, we found that sCD163 related not only to HIV RNA levels but also with CMV IgG titres, and this may be another factor contributing to monocyte activation in our study population. Indeed, animal data suggest acute CMV infection may mediate trafficking of inflammatory monocyte/macrophages from the bone marrow via CCR226.

sCD163 has been previously described as a plasma marker of atherosclerosis in non HIV-infected patients (Aristoteli L P, et al., Atherosclerosis. 2006; 184:342-347; Moreno J A, et al., Atherosclerosis. 2009; 207:103-110). Activated macrophages expressing CD163 have been found in atherosclerotic plaque in non HIV-infected patients (Ratcliffe N R, et al., Immunol Lett. 2001; 77:169-174). These activated macrophages are thought to participate in the atherosclerotic process and contribute to the pathogenesis of cardiovascular disease. However, the specific role played by CD163 itself in the development of atherosclerosis is unknown (Philippidis P, et al., Circ Res. 2004; 94:119-126; Schaer C A, et al., Circ Res. 2006; 99:943-950). In this study, the first among HIV-infected patients to investigate the relationship between sCD163 and atherosclerotic plaque, we find a significant association of sCD163 with the pro-inflammatory CD14+ CD16+ monocyte subsets as well as with sCD14. Additionally, we demonstrate that LPS in plasma, a measure of circulating microbial products that have crossed the gastrointestinal tract, is also elevated in HIV-infected patients in association with sCD163. LPS and bacterial products have been found in chronically HIV infected humans and SIV-infected monkeys and correlate with development of AIDS (Brenchley J M, et al., Nat Med. 2006; 12:1365-1371; Jiang W, et al., J Infect Dis. 2009; 199:1177-1185). LPS is a potent stimulant with other macrophage activation products of CD163 expression and secretion by monocyte/macrophages (Weaver L K, et al., J Leukoc Biol. 2006; 80:26-35; Hintz K A, et al., J Leukoc Biol. 2002; 72:711-717). However, LPS was not itself associated with plaque. In addition, chronicity of HIV infection related to degree of monocyte activation as indicated by sCD163 level. Together these data suggest that HIV-infected individuals are at an increased risk for atherosclerosis due to macrophage-mediated mechanisms, likely resulting from chronic immune activation despite low or undetectable plasma virus.

In contrast to the SMART data, our data in carefully phenotyped patients assessed by coronary angiography, does not suggest a relationship between D-dimer and subclinical atherosclerotic disease. The lack of an association between D-dimer and coronary atherosclerosis in our study may be due to the fact that we investigated early asymptomatic cardiovascular disease before an acute coronary event in contrast to data from the SMART study (Kuller L H, et al., PLoS Med. 2008; 5:e203).

Our study prospectively enrolled a large number of patients with longstanding HIV infection, largely well controlled, with minimal traditional risks and without known coronary disease and a well matched control group of non HIV patients. We used detailed phenotyping of plaque morphology and measurements of monocyte/macrophage activation markers to find a novel relationship with monocyte activation and noncalcified plaque in HIV-infected patients.

Taken together, these data demonstrate monocyte activation in association with increased plaque comprised of predominantly non-calcified plaque in this population. In particular, our data demonstrate that sCD163, a marker of monocyte activation, is strongly and uniquely associated with non-calcified coronary lesions in HIV-infected patients. Development of non-calcified lesions related to persistent monocyte activation may predispose HIV-infected patients to plaque rupture and premature CAD. Our data do not negate the impact and need to modify traditional risk factors, which do appear to be associated with increased calcified lesions.

### Example 3

We have further showed that persistent activation of monocyte-derived cells despite durable virologic suppression in HIV positive individuals on antiretroviral therapy (ART) may contribute to persistent and disabling neurocognitive impairment.

We evaluated whether neural injury (HAND; HIV-associated neurocognitive disorder) in durably virologically suppressed individuals is associated with persistent monocyte activation (as measured by sCD163). We hypothesized that durably virologically suppressed individuals with evidence of greater neural injury (HAND) will show elevated sCD163 compared to virologically suppressed subjects with lesser neural injury (no HAND).

We examined 34 HIV+ patients that had undetectable viral loads and measured sCD163 in plasma. Plasma sCD163 is elevated in HIV+ patients with impaired global deterioration score (GDS) compared to those HIV+ individuals with a normal GDS (P=0.006, Student t test). FIG. 13 shows the results of the comparison demonstrating that increased cCD163 amount is associated with HIV+ individuals with impaired mental function.

Thus, individuals with neurocognitive impairment have higher levels of sCD163 in plasma and elevated monocyte activation despite controlled viral load.

TABLE 3
Demographic and Clinical Characteristics of Study Population by HIV Status

| | | | | | | | | P-value of |
|---|---|---|---|---|---|---|---|---|
| | | | | | P-value | HIV+ on | | HIV+ on |
| | ′HIV− | | | | of all | ART, with | | ART, with |
| | Negative | | All HIV+ | | HIV+ | Undetectable | | Undetectable |
| | Controls | | Subjects | | subjects | Viral Load | | Viral Load |
| | (n = 41) | | (n = 102) | | vs Controls | (n = 69) | | vs Controls |
| Demographics | | | | | | | | |
| Age, y | 44.6 ± 7.6 | | 46.6 ± 6.4 | | 0.10 | 46.8 ± 6.3 | | 0.10 |
| Race, %, | 61/17/7/7/5 | | 63/22/12/1/3 | | 0.19 | 67/19/12/0/3 | | 0.12 |
| White/Black/Hispanic/Asian/Native | | | | | | | | |
| Am | | | | | | | | |
| Family history of premature CHD | 13 | | 22 | | 0.24 | 19 | | 0.46 |
| by NCEP, % | | | | | | | | |
| Framingham risk score | 6.2 ± 5.1 | | 7.8 ± 5.0 | | 0.08 | 7.6 ± 4.9 | | 0.15 |
| Diabetes Mellitus, % | 2 | | 7 | | 0.26 | 7 | | 0.25 |
| Hypertension, % | 12 | | 26 | | 0.06 | 27 | | 0.06 |
| Antihypertensive Medications, % | 8 | | 29 | | 0.003 | 29 | | 0.004 |
| Current Smoker, % | 29 | | 41 | | 0.20 | 40 | | 0.27 |
| Lipid-lowering Medications, % | 13 | | 27 | | 0.05 | 31 | | 0.03 |
| HIV Disease Related Parameters | | | | | | | | |
| Duration Since HIV Diagnosis, y | N/A | | 13.8 ± 6.4 | | N/A | 13.6 ± 6.7 | | N/A |
| Currently on Antiretroviral | N/A | | 95 | | N/A | 100 | | N/A |
| Therapy, % | | | | | | | | |
| Duration of Antiretroviral Therapy, y | N/A | | 7.2 ± 5.0 | | N/A | 7.8 ± 4.6 | | N/A |
| Current PI Treatment, % | N/A | | 52 | | N/A | 49 | | N/A |
| Current NRTI Treatment, % | N/A | | 92 | | N/A | 97 | | N/A |
| Current NNRTI Treatment, % | N/A | | 47 | | N/A | 54 | | N/A |
| CD4+ T-lymphocytes (cells/mm³) | N/A | | 530 ± 287 | | N/A | 544 ± 288 | | N/A |
| (current) | | | | | | | | |
| Nadir CD4+ T-lymphocytes | N/A | | 202 ± 173 | | N/A | 203 ± 175 | | N/A |
| (cells/mm³) | | | | | | | | |
| HIV RNA Viral Load (copies/mL) | N/A | | <50 | (<50, <50) | N/A | <50 | (<50, <50) | N/A |
| Undetectable HIV RNA < | N/A | | 81 | | N/A | 100 | | N/A |
| 50 copies/mL, % | | | | | | | | |
| Traditional CV Risk Factors | | | | | | | | |
| Body mass index, kg/m² | 26.9 ± 4.9 | | 26.3 ± 4.7 | | 0.51 | 26.5 ± 4.3 | | 0.70 |
| Systolic blood pressure, mm Hg | 117 ± 12 | | 120 ± 12 | | 0.23 | 121 ± 11 | | 0.14 |
| Diastolic blood pressure, mm Hg | 76 ± 9 | | 77 ± 9 | | 0.59 | 77 ± 9 | | 0.39 |
| Fasting glucose, mg/dL | 92 ± 10 | | 92 ± 12 | | 0.89 | 93 ± 11 | | 0.75 |
| Total cholesterol, mg/dL | 177 ± 40 | | 180 ± 41 | | 0.77 | 182 ± 42 | | 0.59 |
| HDL-Cholesterol, mg/dL | 48 ± 12 | | 48 ± 15 | | 0.97 | 49 ± 15 | | 0.62 |
| LDL-Cholesterol, mg/dL | 110 ± 33 | | 101 ± 31 | | 0.14 | 101 ± 30 | | 0.17 |
| Triglycerides, mg/dL | 100 ± 58 | | 155 ± 129 | | 0.009 | 158 ± 142 | | 0.01 |
| Creatinine, mg/dL | 1.07 ± 0.15 | | 1.04 ± 0.19 | | 0.35 | 1.02 ± 0.19 | | 0.13 |
| Markers of Monocyte/Macrophage | | | | | | | | |
| Activation | | | | | | | | |
| sCD163, ng/mL | 883 ± 561 | | 1232 ± 716 | | 0.006 | 1172 ± 646 | | 0.02 |
| LPS, ng/mL | 0.08 ± 0.04 | | 0.10 ± 0.04 | | 0.001 | 0.10 ± 0.04 | | 0.002 |
| sCD14, ng/mL | 211 | (121, 374) | 305 | (157, 440) | 0.08 | 296 | (156, 449) | 0.11 |
| Osteopontin, ng/mL | 328 | (269, 455) | 482 | (348, 616) | 0.0002 | 464 | (358, 674) | 0.001 |
| MCP-1, pg/mL | 235 | (190, 299) | 275 | (179, 363) | 0.13 | 281 | (197, 366) | 0.06 |
| Markers of Generalized Inflammation | | | | | | | | |
| and Hemostasis | | | | | | | | |
| hs Interleukin-6, pg/mL, median | 0.6 | (0.5, 1.0) | 0.9 | (0.7, 1.5) | 0.01 | 0.9 | (0.6, 1.3) | 0.05 |
| (IQR) | | | | | | | | |
| C-reactive protein, mg/L, median | 1.3 | (0.7, 3.3) | 1.6 | (0.7, 3.8) | 0.49 | 1.5 | (0.7, 3.8) | 0.66 |
| (IQR) | | | | | | | | |
| D-dimer, ng/mL, median (IQR) | <220 | (<220, 333) | <220 | (<220, 322) | 0.93 | <220 | (<220, 333) | 0.81 |
| Cytomegalovirus IgG titers, median | 36 | (3, 68) | 93 | (65, 194) | <0.0001 | 86 | (64, 194) | <0.0001 |
| (IQR) | | | | | | | | |
| Plaque Characteristics | | | | | | | | |
| Presence of plaque, % | 39 | | 60 | | 0.02 | 61 | | 0.03 |
| Agatston calcium score, median | 0 | (0, 9.9) | 0 | (0, 17.9) | 0.29 | 0 | (0, 22.5) | 0.19 |
| (IQR) | | | | | | | | |
| No. of segments with noncalcified | 0 | (0, 1); | 0 | (0, 1); | 0.049 | 0 | (0, 1); | 0.12 |

| | | | | | |
|---|---|---|---|---|---|
| plaque, n, median (IQR); | 0.46 ± 0.98 | 0.99 ± 1.57 | | 0.84 ± 1.39 | |
| mean ± SD | | | | | |
| Participants with Coronary | 0 | 5 | 0.07 | 3 | 0.17 |
| Stenosis >70%, % | | | | | |

Data reported as mean ± standard deviation (SD) or percentage, except for variables with non-normal distributions, which are reported as median (IQR = interquartile range). CHD indicates coronary heart disease; NCEP, National Cholesterol Education Program; PI, Protease Inhibitor; NRTI, Nucleoside/Nucleotide Reverse Transcriptase Inhibitors; NNRTI, Non-nucleoside Reverse Transcriptase Inhibitors; HDL, High-Density Lipoprotein; LDL, Low-Density Lipoprotein

TABLE 4
Relationships to sCD163 (Pearson and Spearman Correlation Coefficients)

| | | All HIV-Infected | HIV-Infected Patients with |
|---|---|---|---|
| | All patients | Patients | undetectable Viral Load |
| | (n = 143) | (n = 102) | (n = 69) |
| Traditional Risk Factors | | | |
| Age | r = 0.15, p = 0.08 | r = 0.16, p = 0.11 | r = 0.18, p = 0.14 |
| Systolic blood pressure | r = 0.09, p = 0.29 | r = 0.05, p = 0.60 | r = 0.10, p = 0.43 |
| Diastolic blood pressure | r = 0.05, p = 0.54 | r = −0.009, p = 0.93 | r = 0.06, p = 0.62 |
| Total cholesterol | r = −0.11, p = 0.20 | r = −0.14, p = 0.16 | r = −0.007, p = 0.95 |
| LDL-Cholesterol | r = −0.13, p = 0.13 | r = −0.13, p = 0.20 | r = −0.02, p = 0.90 |
| Triglycerides | r = 0.17, p = 0.045 | r = 0.15, p = 0.14 | r = 0.13, p = 0.28 |
| Measurements of Coronary | | | |
| Atherosclerosis | | | |
| Natural Log Segments | r = 0.22, p = 0.01 | r = 0.21, p = 0.04 | r = 0.24, p = 0.046 |
| with Noncalcified | | | |
| Natural Log Calcium | r = −0.04, p = 0.62 | r = −0.06, p = 0.52 | r = 0.05, p = 0.67 |
| Score | | | |
| Monocyte/Macrophage | | | |
| Activation Markers | | | |
| LPS | r = 0.28, p = 0.0007 | r = 0.27, p = 0.007 | r = 0.21, p = 0.08 |
| sCD14 | rho = 0.06, p = 0.46 | rho = 0.22, p = 0.03 | rho = 0.14, p = 0.28 |
| Osteopontin | rho = 0.16, p = 0.06 | rho = 0.03, p = 0.75 | rho = 0.17, p = 0.15 |
| Percentage of | r = 0.20, p = 0.14 | r = 0.35, p = 0.04 | r = 0.29, p = 0.13 |
| CD14+CD16+ monocytes | | | |
| Markers of Generalized | | | |
| Inflammation, Coagulation | | | |
| and CMV | | | |
| hs Interleukin-6 | rho = 0.26, p = 0.005 | rho = 0.27, p = 0.01 | rho = 0.32, p = 0.01 |
| CRP | rho = −0.04, p = 0.59 | rho = −0.03, p = 0.75 | rho = 0.13, p = 0.27 |
| D-dimer | rho = 0.09, p = 0.29 | rho = 0.13, p = 0.18 | rho = 0.16, p = 0.19 |
| Cytomegalovirus IgG | rho = 0.30, p = 0.001 | rho = 0.27, p = 0.01 | rho = 0.23, p = 0.10 |
| titers | | | |
| Markers of HIV Disease | | | |
| Natural Log (Viral Load) | N/A | r = 0.22, p = 0.04 | N/A |
| CD4+ T-lymphocytes | N/A | r = −0.12, p = 0.23 | r = −0.13, p = 0.30 |
| (current) | | | |
| Nadir CD4+ T- | N/A | r = −0.13, p = 0.24 | r = −0.23, p = 0.08 |
| lymphocytes | | | |
| CD4/CD8 ratio | N/A | r = −0.22, p = 0.02 | r = −0.29, p = 0.02 |
| Duration of HIV Infection | N/A | r = 0.19, p = 0.06 | r = 0.23, p = 0.05 |

TABLE 5
Relationships to Plaque Characteristics (Pearson and
Spearman Correlation Coefficient) in All Participants

| | % Noncalcified | Natural Log (Ca |
|---|---|---|
| | plaque | Score +1) |
| Traditional Risk | | |
| Factors | | |
| Age | rho = −0.12, p = 0.32 | r = 0.36, p < 0.0001 |
| Framingham risk score | rho = −0.21, p = 0.08 | r = 0.37, p < 0.0001 |
| Systolic blood pressure | rho = 0.04, p = 0.73 | r = 0.12, p = 0.15 |
| Diastolic blood pressure | rho = 0.19, p = 0.10 | r = 0.12, p = 0.15 |
| Total cholesterol | rho = −0.24, p = 0.04 | r = 0.32, p < 0.0001 |
| LDL-Cholesterol | rho = −0.18, p = 0.14 | r = 0.25, p = 0.003 |
| HDL-Cholesterol | rho = −0.03, p = 0.78 | r = −0.10, p = 0.25 |
| Triglycerides | rho = −0.20, p = 0.09 | r = 0.33, p < 0.0001 |
| Monocyte/Macrophage | | |
| Activation Markers | | |
| sCD163 | rho = 0.23, p = 0.047 | r = −0.04, p = 0.62 |
| LPS | rho = 0.05, p = 0.68 | r = 0.06, p = 0.47 |
| sCD14 | rho = 0.26, p = 0.03 | r = −0.07, p = 0.39 |
| Osteopontin | rho = −0.01, p = 0.94 | r = −0.06, p = 0.47 |
| Markers of Generalized | | |
| Inflammation, | | |
| Coagulation and CMV | | |
| hs Interleukin-6 | rho = 0.17, p = 0.17 | r = 0.03, p = 0.77 |
| CRP | rho = 0.14, p = 0.22 | r = −0.07, p = 0.37 |
| D-dimer | rho = −0.12, p = 0.29 | r = −0.06, p = 0.46 |
| Cytomegalovirus IgG | rho = −0.10, p = 0.44 | r = 0.16, p = 0.09 |
| titers | | |

TABLE 6A
Multivariate Models for Relationship of sCD163 with Noncalcified
Segments and Percent Noncalcified Plaque in All Participants,
Adjusting for Traditional CVD Risk Factors

| | Natural Log Segments | | % | |
|---|---|---|---|---|
| | with Noncalcified | | Noncalcified | |
| | Plaque | | Plaque | |
| | β | | β | |
| | Estimate | p-value | Estimate | p-value |
| HIV status | −0.12 | 0.03 | −4.93 | 0.39 |
| Age (years) | 0.01 | 0.25 | −0.66 | 0.42 |
| Race | −0.14 | 0.60 | −41.73 | 0.18 |
| Ever smoked | 0.03 | 0.61 | 2.73 | 0.60 |
| Diastolic Blood Pressure | 0.03 | 0.13 | 1.05 | 0.03 |
| (mm Hg) | | | | |
| Fasting Glucose (mg/dL) | 0.02 | 0.04 | 0.38 | 0.25 |
| Total cholesterol (mg/dL) | 0.002 | 0.16 | −0.11 | 0.49 |
| HDL-cholesterol (mg/dL) | −0.006 | 0.12 | 0.23 | 0.55 |
| Triglycerides (mg/dL) | −0.001 | 0.02 | −0.06 | 0.14 |
| sCD14 | 0.00005 | 0.67 | 0.01 | 0.17 |
| sCD163 (ng/mL) | 0.0002 | 0.008 | 0.01 | 0.02 |

TABLE 6B
Multivariate Models for Relationship of sCD163 with
Noncalcified Segments in HIV-infected Patients

Natural Log Segments
with Noncalcified Plaque

| | β Estimate | p-value |
|---|---|---|
| Age (years) | 0.01 | 0.13 |
| Race | 0.04 | 0.94 |
| Ever smoked | 0.08 | 0.29 |
| Diastolic Blood Pressure | 0.01 | 0.06 |
| (mm Hg) | | |
| Fasting Glucose (mg/dL) | 0.006 | 0.20 |
| Total cholesterol (mg/dL) | 0.001 | 0.54 |
| HDL-cholesterol (mg/dL) | −0.007 | 0.13 |
| Triglycerides (mg/dL) | −0.001 | 0.08 |
| sCD14 | 0.00009 | 0.49 |
| sCD163 (ng/mL) | 0.0002 | 0.045 |

TABLE 6C
Multivariate Models for Relationship of sCD163 with
Noncalcified Segments in HIV-Seronegative Controls

Natural Log Segments
with Noncalcified Plaque

| | β Estimate | p-value |
|---|---|---|
| Age (years) | −0.0001 | 0.99 |
| Race | −0.38 | 0.09 |
| Ever smoked | −0.18 | 0.02 |
| Diastolic Blood Pressure | 0.02 | 0.03 |
| (mm Hg) | | |
| Fasting Glucose (mg/dL) | −0.006 | 0.31 |
| Total cholesterol (mg/dL) | 0.007 | 0.002 |
| HDL-cholesterol (mg/dL) | −0.008 | 0.18 |
| Triglycerides (mg/dL) | −0.005 | 0.01 |
| sCD14 | −0.0004 | 0.04 |
| sCD163 (ng/mL) | 0.00001 | 0.89 |

1. (canceled)

2. An assay for determining or monitoring the effectiveness of a treatment of a macrophage-mediated disease in an individual infected with HIV, comprising the steps of:
(a) contacting a first biological sample, wherein the first biological sample comprises plasma, blood or cerebral spinal fluid obtained from an individual infected with HIV and further infected with a macrophage-mediated disease prior to administering a treatment with an antibody against soluble CD163;
(b) measuring the amount of the soluble CD163 in the first biological sample;
(c) administering the treatment to the patient;
(d) contacting a second biological sample, wherein the second biological sample comprises plasma, blood, or cerebral spinal fluid from the individual obtained after administration of the treatment with an antibody against the soluble CD163;
(e) measuring the amount of the soluble CD163 in the second biological sample; and
(f) comparing the difference in the amount of the soluble CD163 between the first and the second biological sample, wherein the treatment is effective if the amount of the soluble CD163 in the second biological sample is decreased by at least 10% compared to the first biological sample.

3. The method of claim 2, further comprising administering to the individual a different treatment or increased dosage of the same treatment if the soluble CD163 is not decreased by at least 10% in the second biological sample.

4. (canceled)

5. (canceled)

6. (canceled)

7. The assay of claim 2, wherein the macrophage-mediated disease is selected from the group consisting of: AIDS-related dementia, peripheral neuropathy, and HIV-associated heart disease.

8. The assay of claim 2, further comprising the step of depleting the first and/or the at least second biological sample from monocytes.

9. The assay of claim 2, wherein the individual has detectable HIV levels.

10. The assay of claim 2, wherein the second biological sample is obtained at least 3 months week after the first biological sample.

11. The assay of claim 2, wherein the second biological sample is obtained at least 6 months after the first biological sample.

12. The assay of claim 2, wherein the individual is a mammalian model.

13. The assay of claim 12, wherein the mammalian model is a primate model or a human.

14. The assay of claim 2, wherein the measuring step is performed using an ELISA technique.

15. The assay of claim 2, wherein the antibody does not measure macrophage-associated CD163.

16. The assay of claim 2, wherein the second biological sample is obtained at least 9 months after the first biological sample.

17. A method for determining time of onset for administering treatment to an individual infected with HIV prior to appearance of clinical symptoms, the method comprising the steps of:
(a) contacting a first biological sample, wherein the first biological sample comprises plasma, blood, or cerebral spinal fluid obtained from the individual with an antibody against soluble CD163;
(b) measuring the amount of the soluble CD163 in the first biological sample;
(c) comparing the amount of the soluble CD163 in the first biological sample to a reference;
(d) administering a treatment to the individual if the amount of the soluble CD163 is increased by at least 10% in the first biological sample compared to the reference.

18. The method of claim 17, wherein the reference is a second biological sample, wherein the second biological sample comprises plasma, blood, or cerebral spinal fluid obtained from the individual at a time point that is subsequent to obtaining the first biological sample.

19. (canceled)

20. A computer system for obtaining data from at least one monocyte-depleted sample comprising plasma obtained from at least one individual, the system comprising:
(a) a specimen container to hold the at least one sample;
(b) a determination module configured to determine read-out information, wherein said read-out information comprises information representing an amount of soluble CD163 in the at least one sample;
(c) a storage device configured to store data output from said determination module;
(d) comparison module adapted to compare the data obtained from the determination module with reference data on said storage device, whereby a change in the level of the soluble CD 163 is determined; and
(e) a display module for displaying retrieved content to the user, wherein the retrieved content comprises an increase, decrease or value for the soluble CD163 in the at least one monocyte-depleted sample compared to a reference sample.

21. The computer system of claim 20, wherein the reference sample is a different monocyte-depleted sample from the same individual, from a different individual, or from a population of individuals infected with HIV.

22. The computer system of claim 20, wherein the reference sample is a numeric value.

23. The assay of claim 7, wherein the HIV-associated heart disease is coronary atherosclerosis.

24. The assay of claim 7, wherein the individual is infected with HIV has not undergone seroconversion.

25. The method of claim 17, wherein the individual is infected with HIV has not undergone seroconversion.
